(19) United States
(12) Patent Application Publication
(10) Pub. No.: US 2025/0102508 A1
(43) Pub. Date: Mar. 27, 2025

(54) LEUKEMIA-RELATED MARKER AND USE THEREOF

(71) Applicant: Dalian University of Technology, Dalian, Liaoning (CN)

(72) Inventor: Changhao Cui, Panjin, Liaoning (CN)

(73) Assignee: Dalian University of Technology, Dalian, Liaoning (CN)

(21) Appl. No.: 18/724,696

(22) PCT Filed: Dec. 31, 2021
(86) PCT No.: PCT/CN2021/143649

§ 371 Date: Jun. 27, 2024

(51) Int. Cl. G01N 33/574; A61K 31/713; A61K 38/21; A61K 39/00; A61P 35/02; C07K 16/32; G01N 33/50

(52) U.S. Cl. CPC G01N 33/57426; A61K 31/713; A61K 38/217; A61P 35/02; C07K 16/32; G01N 33/5073; A61K 2039/505; C07K 2317/732; G01N 2800/7028

(57) ABSTRACT

The invention relates to biomarker Allergin-1 specifically expressed by leukemia stem cells and use thereof. Specifically, the invention relates to methods for identification of LSCs, diagnosis, disease course monitoring and prognosis of leukemia (especially AML), or determination of risk of developing leukemia (especially AML), or determination of the presence of severe leukemia (especially AML) by detecting expression or activity of the biomarker Allergin-1 in a subject or a sample from the subject, in which Allergin-1 is specifically and abnormally highly expressed in LSCs. The invention also provides methods for treating leukemia (especially AML) in a subject by inhibiting/antagonizing expression/activity of Allergin-1.

### FIELD OF THE INVENTION

The invention relates to biomarker Allergin-1 specifically expressed by leukemia stem cells (LSCs) and use thereof. Specifically, the invention relates to diagnostic products, therapeutic medicaments or compositions for leukemia, especially acute myeloid leukemia (AML), application of detection agents for detecting the marker Allergin-1 in the manufacture of products for diagnosis, disease course monitoring and prognosis of leukemia, and use of the agents in the manufacture of medicaments for treatment of leukemia.

Additionally the invention relates to methods for identification of LSCs, diagnosis, disease course monitoring and prognosis of leukemia (especially AML), or determination of risk of developing leukemia (especially AML), or determination of severe leukemia (especially AML) by detecting expression or activity of biomarker Allergin-1 in a subject or a sample from a subject, in which Allergin-1 is specifically and abnormally highly expressed in LSCs. The invention also provides methods for treating leukemia (especially AML) in a subject by inhibiting/antagonizing expression/activity of Allergin-1. In addition, provided is a kit and array comprising a tool, particularly an antibody, for detection of a biomarker specifically expressed by leukemia stem cells.

### BACKGROUND OF THE INVENTION

Acute myeloid leukemia (AML) is a malignant disease caused by blocking of the differentiation and infinite proliferation of hematopoietic stem cells (HSCs). The disease is dangerous, easy to relapse, and has a high fatality rate and increasing incidence rate year after year (Dores G M, Devesa S S, Curtis R E, Linet M S, Morton L M: Acute leukemia incidence and patient survival among children and adults in the United States, 2001-2007. Blood 2012, 119(1):34-43). With the increasing incidence of leukemia and the aging of the global population, AML accounts for an increasing proportion of malignant tumors, becoming a major threat to human health. In terms of basic and clinical research on AML, although scholars at home and abroad have paid a great deal of effort and progress has been made, AML patients still face many problems such as a high recurrence rate and low long-term survival rate of more than 5 years, in particular, there is about 60%-80% recurrence rate among patients aged >60 years old (Dombret H, Gardin C: An update of current treatments for adult acute myeloid leukemia. Blood 2016, 127(1):53-61; Short N J, Konopleva M, Kadia T M, Borthakur G, Ravandi F, DiNardo C D, Daver N: Advances in the Treatment of Acute Myeloid Leukemia: New Drugs and New Challenges. Cancer Discov 2020, 10(4):506-525). In recent years, emerging cell therapy and immunotherapy have achieved good results in the treatment of lymphoid leukemia, but they are difficult to implement in AML, since targeted treatment of AML has higher requirements for the choice of the target. With the continuous in-depth study of the molecular mechanism of tumorigenesis, molecular targeted therapy has become a breakthrough to overcome tumors. Therefore, further exploration of effective molecular targets for the treatment of AML has become an important scientific subject in recent AML research (Sami S A, Darwish N H E, Barile A N M, Mousa S A: Current and Future Molecular Targets for Acute Myeloid Leukemia Therapy. Curr Treat Option On 2020, 21(1)).

It has been shown by studies that leukemia stem cells (LSCs) play a key role in the onset, development and recurrence of AML (Ishikawa F, Yoshida S, Saito Y, Hijikata A, Kitamura H, Tanaka S, Nakamura R, Tanaka T, Tomiyama H, Saito N et al: Chemotherapy-resistant human AML stem cells home to and engraft within the bone-marrow endosteal region. Nat Biotechnol 2007, 25(11):1315-1321; Saito Y, Yuki H, Kuratani M, Hashizume Y, Takagi S, Honma T, Tanaka A, Shirouzu M, Mikuni J, Handa N et al: A Pyrrolo-Pyrimidine Derivative Targets Human Primary AML Stem Cells in Vivo. Sci Transl Med 2013, 5(181); Pabst C, Krosl J, Fares I, Boucher G, Ruel R, Marinier A, Lemieux S, Hebert J, Sauvageau G: Identification of small molecules that support human leukemia stem cell activity ex vivo. Nat Methods 2014, 11(4):436-442). In 1994, Lapido et al. firstly isolated a CD34⁺CD38⁻ LSC subpopulation from bone marrow cells of AML patients, and confirmed that this subpopulation has a powerful self-renewal and proliferation ability like HSCs (Lapidot T, Sirard C, Vormoor J, Murdoch B, Hoang T, Cacerescortes J, Minden M, Paterson B, Caligiuri M A, Dick J E: A Cell Initiating Human Acute Myeloid-Leukemia after Transplantation into Scid Mice. Nature 1994, 367(6464):645-648). More than 96% of the cells in this subpopulation are in the GO phase, which can escape the killing effect of chemical drugs, leading to drug resistance and easy recurrence (Ishikawa F, Yoshida S, Saito Y, Hijikata A, Kitamura H, Tanaka S, Nakamura R, Tanaka T, Tomiyama H, Saito N et al: Chemotherapy-resistant human AML stem cells home to and engraft within the bone-marrow endosteal region. Nat Biotechnol 2007, 25(11):1315-1321). In 2014, John E. Dick et al. found that 25% of AML patients have a mutation in the DNMT3a gene that leads to formation of pre-LSCs, which have similar function to normal HSCs but grow abnormally. These pre-LSCs present in bone marrow are not sensitive to chemotherapy and other therapies, and may eventually undergo other changes that lead to the recurrence of AML (Shlush L I, Zandi S, Mitchell A, Chen W C, Brandwein J M, Gupta V, Kennedy J A, Schimmer A D, Schuh A C, Yee K W et al: Identification of pre-leukaemic haematopoietic stem cells in acute leukaemia. Nature 2014, 508(7496):420-420). The above studies have shown that LSCs are the main cause of the onset, development and recurrence of AML. Therefore, exploring and discovering new molecular targets that effectively inhibit the activity of LSCs to develop corresponding therapeutic drugs is expected to fundamentally achieve the ultimate goal of controlling leukemia.

A large number of studies have shown that cell surface molecules play an important role in the activity of LSCs and the onset and development of AML (Zheng J K, Umikawa M, Cui C H, Li J Y, Chen X L, Zhang C Z, Huynh H, Kang X L, Silvany R, Wan X et al: Inhibitory receptors bind ANGPTLs and support blood stem cells and leukaemia development. Nature 2012, 488(7413):684-684; Majeti R, Chao M P, Alizadeh A A, Pang W W, Jaiswal S, Gibbs K D, van Rooijen N, Weissman I L: CD47 Is an Adverse Prognostic Factor and Therapeutic Antibody Target on Human Acute Myeloid Leukemia Stem Cells. Cell 2009, 138(2):286-299; van Rhenen A, van Dongen G A M S, Kelder A, Rombouts E J, Feller N, Moshaver B, Stigter-van Walsum M, Zweegman S, Ossenkoppele G J, Schuurhuis G J: The novel AML stem cell-associated antigen CLL-1 aids in discrimination between normal and leukemic stem cells. Blood 2007, 110(7):2659-2666; Kikushige Y, Shima T, Takayanagi S, Urata S, Miyamoto T, Iwasaki H, Takenaka K, Teshima T, Tanaka T, Inagaki Y et al: TIM-3 Is a Promising Target to Selectively Kill Acute Myeloid Leukemia Stem Cells. Cell Stem Cell 2010, 7(6):708-717; Kang X L, Lu Z G, Cui C H, Deng M, Fan Y Q, Dong B J, Han X, Xie F C, Tyner J W, Coligan J E et al: The ITIM-containing receptor LAIR1 is essential for acute myeloid leukaemia development. Nat Cell Biol 2015, 17(5):665-U286), and cell surface molecules that are specifically expressed in LSCs can be used as targets for the targeted treatment of AML. The study of targeted treatment of AML with cell surface molecules as targets has a very important practical clinical significance. On one hand, cell surface molecules specifically expressed in LSCs can be used to monitor the course of AML and determine the prognosis; on the other hand, cell surface molecules specifically expressed in LSCs can be used as targets to develop new therapies that are more effective and even targeted to cure AML. In recent years, with the in-depth research on LSCs, it has been found that the differential expression of cell surface molecules such as CD33 (Hauswirth A W, Florian S, Printz D, Sotlar K, Krauth M T, Fritsch G, Schernthaner G H, Wacheck V, Selzer E, Sperr W R et al: Expression of the target receptor CD33 in CD34(+)/CD38(−)/CD123(+) AML stem cells. Eur J Clin Invest 2007, 37(1):73-82), CD47 (Majeti R, Chao M P, Alizadeh A A, Pang W W, Jaiswal S, Gibbs K D, van Rooijen N, Weissman I L: CD47 Is an Adverse Prognostic Factor and Therapeutic Antibody Target on Human Acute Myeloid Leukemia Stem Cells. Cell 2009, 138(2):286-299), CD96 (Hosen N, Park C Y, Tatsumi N, Oji Y, Sugiyama H, Gramatzki M, Krensky A M, Weissman I L: CD96 is a leukemic stem cell-specific marker in human acute myeloid leukemia. P Natl Acad Sci USA 2007, 104(26):11008-11013), CD123 (Hosen N, Park C Y, Tatsumi N, Oji Y, Sugiyama H, Gramatzki M, Krensky A M, Weissman I L: CD96 is a leukemic stem cell-specific marker in human acute myeloid leukemia. P Natl Acad Sci USA 2007, 104(26):11008-11013; Hwang K, Park C J, Jang S, Chi H S, Kim D Y, Lee J H, Lee J H, Lee K H, Im H J, Seo J J: Flow cytometric quantification and immunophenotyping of leukemic stem cells in acute myeloid leukemia. Ann Hematol 2012, 91(10):1541-1546; Abdel-Wahab O, Mullally A, Hedvat C, Garcia-Manero G, Patel J, Wadleigh M, Malinge S, Yao J J, Kilpivaara O, Bhat R et al: Genetic characterization of TET1, TET2, and TET3 alterations in myeloid malignancies. Blood 2009, 114(1):144-147), TIM-3 (Kikushige Y, Shima T, Takayanagi S, Urata S, Miyamoto T, Iwasaki H, Takenaka K, Teshima T, Tanaka T, Inagaki Y et al: TIM-3 Is a Promising Target to Selectively Kill Acute Myeloid Leukemia Stem Cells. Cell Stem Cell 2010, 7(6):708-717) and CLL-1 (van Rhenen A, van Dongen G A M S, Kelder A, Rombouts E J, Feller N, Moshaver B, Stigter-van Walsum M, Zweegman S, Ossenkoppele G J, Schuurhuis G J: The novel AML stem cell-associated antigen CLL-1 aids in discrimination between normal and leukemic stem cells. Blood 2007, 110(7):2659-2666) can be used to differentiate LSCs from HSCs. However, the above cell surface molecules are either weakly expressed or partially expressed in LSCs, or also partially expressed in HSCs, which makes them have certain limitations as therapeutic targets. Therefore, it is necessary to further explore more effective cell surface molecules as new target molecules.

Allergin-1, also known as MILR1, is an immunosuppressive receptor belonging to the immunoglobulin-like receptor superfamily. It is mainly expressed in myeloid cells such as macrophages, neutrophils and mast cells in humans and mice. Allergin-1 is involved in a variety of pathophysiological processes in the body (Hitomi K, Tahara-Hanaoka S, Someya S, Fujiki A, Tada H, Sugiyama T, Shibayama S, Shibuya K, Shibuya A: An immunoglobulin-like receptor, Allergin-1, inhibits immunoglobulin E-mediated immediate hypersensitivity reactions. Nat Immunol. 2010, 11(7):601-607). However, it has not been reported on the expression characteristics of Allergin-1 in LSCs and AML cells, the biological effects of Allergin-1 in the onset and development of AML, and the study of the feasibility of disease course monitoring, prognosis, targeted therapy and immunotherapy of AML using Allergin-1 as the biomarker or molecular target.

Therefore, it is urgent to thoroughly study the biological effects of Allergin-1 as a new biomarker on leukemia to obtain methods and systems for highly specific diagnosis of leukemia, as well as related targets, methods and medicaments for targeted therapy and immunotherapy.

### SUMMARY OF THE INVENTION

For the above technical problems, we found that: Allergin-1 is not expressed in normal HSCs, but specifically and abnormally highly expressed in LSCs and monocytic AML cells; Allergin-1 has an expression level in a patient with M4 and M5 subtypes of AML that is negatively correlated with survival rate of the patient, and especially, is specifically and abnormally expressed in the M5 subtype with extremely low survival rate; the self-renewal ability of Allergin-1⁺ LSCs is significantly higher than that of Allergin-1⁻ LSCs; the expression level of late differentiation marker of late myeloid cells Allergin-1⁺ AML cells, CD11b, is significantly lower than that of Allergin-1⁻ AML cells; IFN-γ promotes the proliferation of AML cells and the expression level of Allergin-1; knocking down Allergin-1 significantly inhibits the proliferation of AML cells, significantly promotes the expression level of CD11b (a later marker for a more differentiated myeloid cells) of AML cells, and significantly inhibits in vivo engraftment (or in vivo growth) of AML cells; the cytotoxicity of effector cells to AML cells mediated by the Allergin-1 antibody is stimulated in vitro; Allergin-1 antibody can mediate the elimination of root LSCs and monocytic AML cells of AML by effector cells in vivo; and Allergin-1⁻ AML cells promote the expression level of CD25 (the marker of activated NK cells) of normal human peripheral blood monocyte-derived CD3⁻CD56⁺ NK cells, and promote the cytotoxicity of CD3⁻CD56⁺ NK cells. On this basis, Allergin-1 is used as a biomarker for identification of LSCs, diagnosis, disease course monitoring and prognosis of leukemia (especially AML), and as a target for targeted therapy and/or immunotherapy of leukemia, especially AML.

In one aspect of the invention, the invention relates to methods for identification of LSCs by detecting expression or activity of biomarker Allergin-1 in a subject or a sample from the subject, in which Allergin-1 is specifically and abnormally highly expressed in LSCs.

In one aspect of the invention, the invention relates to methods for diagnosis, disease course monitoring and prognosis of leukemia (especially AML), or determination of risk of developing leukemia (especially AML), or determination of severity of leukemia (especially AML) by detecting expression or activity of biomarker Allergin-1 in a subject or a sample from the subject, in which the expression level of Allergin-1 in AML patients with M4 and M5 subtypes is negatively correlated with survival rate of patients, and Allergin-1 is abnormally highly expressed in LSCs and monocytic AML cells. In some embodiments, Allergin-1 is highly expressed in AML patients, and specifically and abnormally highly expressed especially in the M5 subtype.

In one aspect of the invention, the invention relates to a method for identification of LSCs, comprising the identification of LSCs by detecting expression or activity of the biomarker Allergin-1 in a subject or a sample from the subject;

Optionally, by detecting expression or activity of the biomarker Allergin-1 in hematopoietic stem cells in the sample.

Optionally, expression level or activity of Allergin-1 in hematopoietic stem cells is significantly higher than that in normal hematopoietic stem cells, which is an indication that the hematopoietic stem cells are LSCs.

In the above method, the expression level or activity of Allergin-1 in hematopoietic stem cells is at least about 3 times that of normal hematopoietic stem cells, which is an indication that the hematopoietic stem cells are LSCs. Preferably, the expression level or activity of Allergin-1 in hematopoietic stem cells is about 3 times to about 100 times, more preferably about 10-90 times, about 20-80 times, about 30-70 times, such as 40 times, 45 times, 50 times, 55 times, 60 times of that in normal hematopoietic stem cells.

In one aspect of the invention, the invention relates to use of an agent for detecting expression level or activity of Allergin-1 in a sample in the manufacture of a product for identifying LSCs.

In one aspect of the invention, the invention relates to use of an agent for detecting expression level or activity of Allergin-1 in a sample in the manufacture of a product for diagnosis, disease course monitoring and prognosis of leukemia (especially AML).

Preferably, the agent for detecting the expression level or activity of Allergin-1 in the sample is combined with one or more other agents.

More preferably, the one or more other agents are IFN-7.

In one aspect of the invention, the invention relates to use of an agent for detecting combination of Allergin-1 and at least one biomarker in the manufacture of a product for identifying LSCs. In some embodiments, the combination of Allergin-1 and at least one biomarker is Allergin-1⁺CD34⁺CD38⁻.

In one aspect of the invention, the invention relates to use of an agent for detecting combination of Allergin-1 and at least one biomarker in the manufacture of a product for diagnosis, disease course monitoring and prognosis of leukemia (especially AML). In some embodiments, the combination of Allergin-1 and at least one biomarker is Allergin-1⁺CD34⁺CD38⁻.

Preferably, the agents described above include but are not limited to agents used for detecting the expression level of Allergin-1 by RT-PCR, real-time quantitative PCR, immunoassay (such as ELISA, RIA, multiple immunoassay, immunofluorescence assay, western blot, or dot blot), in situ hybridization or chip technology. More preferably, the agents include primers/probes for Allergin-1 gene, molecular beacons or antibodies and/or ligands against Allergin-1 protein, and small molecular compounds.

Preferably, the products described above include but are not limited to chips, agents, test papers, formulations, kits or high-throughput screening platforms.

In one aspect of the invention, the invention relates to use of a modulator (inhibitor/antagonist) for expression or activity of Allergin-1 in the manufacture of a product for treatment of leukemia (especially AML) in a subject. In some embodiments, the treatment is molecular immunotherapy.

In one aspect of the invention, the invention provides a medicament or composition for treatment of leukemia (especially AML), comprising an inhibitor/antagonist capable of inducing cytotoxicity of immune effector cells to AML cells against Allergin-1 molecule and/or functional expression of Allergin-1, and/or other pharmaceutically acceptable carriers and/or adjuvants compatible with the inhibitor.

The suitable inhibitor and/or antagonist described above refers to any substance that can reduce the expression of nucleic acid encoding Allergin-1, reduce the level of Allergin-1 protein, or inhibit the activity of Allergin-1, for example, substances that reduce the activity of Allergin-1 protein, reduce the stability of Allergin-1 gene or protein, down-regulate the expression of Allergin-1, reduce the effective acting time of Allergin-1 protein, or inhibit the transcription and translation of Allergin-1.

Preferably, the inhibitor and/or antagonist described above include, but are not limited to: nucleic acid inhibitor, protein inhibitor, proteolytic enzyme, protein binding molecule; and combination thereof. Among them, the nucleic acid inhibitor is selected from interfering molecules that target Allergin-1 or its transcript and can inhibit expression or transcription of Allergin-1 gene, including shRNA (small hairpin RNA), small interfering RNA (siRNA), dsRNA, microRNA, or constructs capable of expressing or forming the shRNA, siRNA, dsRNA, microRNA. The protein binding molecule is selected from substances that specifically bind to Allergin-1 protein, such as antibodies or ligands capable of inhibiting the activity of Allergin-1 protein; competitors for Allergin-1 ligand binding site, including Allergin-1 receptors and ligand binding fragments thereof, soluble truncated Allergin-1 receptors, soluble Allergin-1 receptor fusion proteins, e.g. Allergin-1 fusion proteins or ligand fusion proteins containing Fc portion of IgG immunoglobulins; peptidomimetics; peptide inhibitors; small molecule compounds; and combination thereof.

More preferably, the inhibitor and/or antagonist is shRNA; especially preferably, the shRNA has the sequence shown in SEQ ID NO: 12, 13, 14, 15, 16 or 17.

More preferably, the inhibitor and/or antagonist is an antibody that specifically binds to Allergin-1; preferably, for example, the specific antibody includes a monoclonal antibody, polyclonal antibody, neutralizing antibody, antigen-binding fragment, and conjugated Allergin-1 antibody.

In one aspect of the invention, the invention provides a product for diagnosis, disease course monitoring and prognosis of leukemia (especially AML). The product includes a chip, a formulation or a kit for detecting expression level of Allergin-1. Further, the kit includes at least one pair of primers for specifically amplifying Allergin-1 gene. Preferably, the primer has the sequence shown in SEQ ID NO: 18, 19, 20, 21, 22 or 23.

In one aspect of the invention, the invention provides a combination of biomarkers, comprising Allergin-1, CD34 and CD38.

In one aspect of the invention, the invention provides use of the combination of biomarkers Allergin-1, CD34 and CD38 for identification of LSC. According to a specific embodiment of the invention, Allergin-1 positive, CD34 positive, and CD38 negative in the biomarkers are the indicators of LSCs.

In one aspect of the invention, the invention provides an inhibitor/antagonist capable of inducing cytotoxicity of immune effector cells to AML or reducing functional expression of AML cells, such as Allergin-1 monoclonal antibody, Allergin-1 neutralizing antibody, Allergin-1 antibody conjugated to anti-tumor drug, antigen-binding fragment, and small molecule compound.

In one aspect of the invention, the invention provides a method for improving activity or immunotherapy effect of NK cells by administering to a subject an effective amount of an inhibitor or antagonist for Allergin-1 expression or activity.

In one aspect of the invention, the invention provides a method for screening candidate drugs for treatment of leukemia (especially AML) by biomarker Allergin-1. Preferably, the method includes treating a system expressing or containing Allergin-1 gene with a substance to be screened; and detecting expression level of the Allergin-1 gene in the system; wherein, if the substance to be screened can reduce the expression level of the Allergin-1 gene, the substance to be screened is identified to be a candidate drug for treatment of leukemia (especially AML).

More preferably, the system described above includes (but is not limited to): cell system, subcellular system, solution system, tissue system, organ system or animal system. The candidate drug includes (but is not limited to): interfering molecules designed for Allergin-1 gene or upstream or downstream genes thereof, nucleic acid inhibitors, small molecule compounds.

### DESCRIPTION OF THE DRAWINGS

FIG. 1 is diagrams showing the expression of Allergin-1 detected in AML patients, in which:

FIG. 1A shows that the expression level of Allergin-1 is negatively correlated with survival rate of AML patients;

FIG. 1B shows that Allergin-1 is abnormally highly expressed in AML patients with M5 subtype;

FIG. 2 is diagrams showing the expression of Allergin-1 detected in healthy cord blood-HSCs and LSCs of patients with primary AML, in which:

FIGS. 2A and 2C show the expression of Allergin-1 in CD34⁺CD38⁻, CD34⁺CD38⁺ and CD34⁻ cells of healthy human cord blood;

FIGS. 2B and 2D show the expression of Allergin-1 in CD34⁺CD38⁻, CD34⁺CD38⁺ and CD34⁻ cells of bone marrow of patients with primary AML;

FIG. 2E shows the expression of Allergin-1 in monocytes of healthy human and patients with primary AML;

FIG. 3 is diagrams showing the self-renewal ability detected for LSCs that are positive for Allergin-1;

FIG. 4 is diagrams showing the correlation between the expression of Allergin-1 detected and the proliferation and differentiation of AML cells, in which:

FIGS. 4A and 4B show that the proliferation ability of Allergin-1 positive AML cells is significantly higher than that of Allergin-1 negative AML cells;

FIGS. 4C and 4D show that the expression level of CD11b in Allergin-1 positive AML cells is significantly lower than that in Allergin-1 negative AML cells;

FIG. 5 is diagrams showing the interfering effect of shRNA on the expression of Allergin-1, in which:

FIGS. 5A and 5B show the knockdown efficiency of Thp-1 (A) and U937 (B) cells detected by Western blot;

FIGS. 5C and 5D show that knockdown of Allergin-1 inhibits the proliferation of Thp-1 (C) and U937 (D) cells;

FIGS. 5E and 5F show that knockdown of Allergin-1 inhibits the expression level of CD11b in Thp-1 (E) and U937 (F) cells;

FIGS. 5G and 5H show the effect of knockdown of Allergin-1 on morphology of Thp-1(G) and U937(H) cells detected by Wright-Giemsa staining;

FIG. 6 is diagrams showing the effect of IFN-7 on the expression level of Allergin-1 and proliferation of AML cells, in which:

FIGS. 6A and 6B show that IFN-7 promotes the proliferation of AML cells (FIG. 6A) and the expression level of Allergin-1 (FIG. 6B);

FIG. 6C shows that the proliferation of Allergin-1⁺ AML cells pretreated with IFN-7 is significantly higher than that of control Allergin-1⁻ AML cells;

FIG. 7 is diagrams showing that knockdown of Allergin-1 inhibits the engraftment of AML cells in mice, in which:

FIG. 7A shows that the weight of liver and spleen of mice transplanted with Thp-1 cells with knockdown of Allergin-1 is significantly lower than that of the control group;

FIG. 7B shows that the engraftment ability of Thp-1 cells with knockdown of Allergin-1 in liver and spleen cells of mice is significantly lower than that of control cells;

FIG. 8 is diagrams showing Allergin-1 antibody-dependent effector cell cytotoxicity (ADCC) on AML cells, in which:

The left panel shows the expression of Allergin-1 in Thp-1 cells detected by flow cytometry; the right panel shows that the cytotoxicity of human peripheral blood mononuclear cells (PBMCs) on Thp-1 cells mediated by the Allergin-1 antibody is significantly higher than that of the IgG1 antibody in control group;

FIG. 9 is diagrams showing the effect of the Allergin-1 antibody on eliminating bone marrow cells of patients with primary AML in NOD/SCID mice in vivo, in which,

FIG. 9A is a schematic diagram of a human-mouse xenograft model mouse transplanted with bone marrow cells of patients with primary AML treated with the Allergin-1 antibody;

FIG. 9B shows that the proportion of human CD45⁺ cells in mouse liver cells in the Allergin-1 antibody treatment group is significantly less than that in the control IgG1 antibody treatment group;

FIGS. 9C-9D show that the proportion of human CD45⁺ cells (upper left panel in D), CD34⁺ cells in human CD45⁺ cells (upper panel in C, upper right panel in D), and CD34⁺CD38⁻ cells in human CD45⁺ cells (bottom panel in C, bottom left panel in D) and Allergin-1⁺ cells in human CD45⁺ cells (middle panel in C, bottom right panel in D) in the mouse bone marrow cells in the Allergin-1 antibody treatment group is significantly less than that in the control IgG1 antibody treatment group;

FIG. 10 is diagrams showing the effect of Allergin-1 antibody on the hematopoietic reconstitution ability of CD34 positive cells from healthy human cord blood in NOD/SCID mice in vivo, in which:

FIG. 10A is a schematic diagram of a human-mouse xenograft model mouse transplanted with CD34⁺ cells from healthy human cord blood treated with the Allergin-1 antibody;

FIGS. 10B-10D show that there is no significant difference in the proportion of human CD45⁺ cells (FIG. 10B) and CD19⁺ or CD33⁺ cells in human CD45⁺ cells (FIG. 10C) in the mouse bone marrow cells between the Allergin-1 antibody treatment group and the control IgG1 antibody treatment group; the proportion of Allergin-1⁻CD33⁺ cells in human CD45⁺ cells in the mouse bone marrow in the Allergin-1 antibody treatment group is significantly reduced compared to that in the control IgG1 antibody treatment group (FIG. 10D);

FIG. 11 is diagrams showing the effect of Allergin-1 on inhibiting the activity and cytotoxicity of NK cells derived from peripheral blood mononuclear cells of healthy human;

FIGS. 11A-D show that the expression level of CD25 (FIGS. 11A-C) and cytotoxicity (FIG. 11D) of CD3⁺CD56⁺ NK cells co-cultured with Allergin-1⁻ Thp-1 cells are significantly higher than that of CD3⁺CD56⁺ NK cells co-cultured with Allergin-1⁺ Thp-1 cells;

FIG. 12 is the sequence of Allergin-1 gene;

FIG. 13 is the bone marrow sample information of the patients with primary AML used in examples of the present invention;

FIG. 14 is shRNA sequences targeting Allergin-1 gene; and

FIG. 15 is primer sequences for amplifying Allergin-1 gene.

### DETAILED DESCRIPTION OF THE INVENTION

### Definition

Unless otherwise specified, all technical and scientific terms used herein have the same meaning as commonly understood by those skilled in the art to which the present invention belongs, but in case of conflict, the definition in this specification shall prevail.

As used in the specification and claims, the singular forms “a”, “an” and “the (said)” include plural forms, unless indicated otherwise in the context clearly.

Unless otherwise specified, the percentages (%) in the specification are weight percentages (weight %).

All numerical values or expressions related to the amount of components, process conditions, etc. used in the specification and claims should be understood to be modified by “about” in all cases. When the term “about” refers to a range of quantity or values, it means that the range of quantity or values referred to is an approximation within the experimental variability (or within the error of a statistical experiment). Thus, the range of the quantity or values can vary between, e.g., ±5 of the range of the quantity or values.

All ranges relating to the same components or properties include endpoints, which can be independently combined. Since these ranges are continuous, they include every value between the minimum and maximum values. It should also be understood that any numerical range used in the present application is intended to include all sub-ranges within the range.

When physical properties such as molecular weight or chemical properties are defined in a range in the present invention, all combinations and sub-combinations of the range and specific embodiments therein shall be included. The term “comprising” (and related terms such as “containing” or “contain” or “having” or “including”) includes such embodiments that the embodiment is, for example, any combination of substances, compositions, methods, or processes, which “consists of the described features” or “essentially consists of the described features”.

The “and/or” used in the specification and claims should be understood as the “either or both” of the related components, that is, the components coexist in some cases and exist separately in other cases. Multiple components listed with “and/or” should be understood in the same way, that is, “one or more” related components. In addition to the components specifically identified in the “and/or” clause, other components may optionally be present, regardless of the correlation to those specifically identified components. Therefore, as a non-limiting example, referring to “A and/or B”, when used to link open-ending words such as “include”, it may only refer to A (optionally including components other than B) in one embodiment; may only refer to B (optionally including components other than A) in another embodiment; refers to A and B (optionally including other components) in yet another embodiment; and the like.

It should be understood that, unless explicitly indicated to the contrary, in any method claimed herein including more than one steps or acts, the order of the steps and acts of the method need not be limited to the described order of the steps and acts of the method.

The abbreviations used in the present invention have the usual meanings in the fields of chemistry, biology and pharmacy.

The term “effective amount” or “therapeutically effective amount” refers to the amount of an agent sufficient to provide the desired biological result. The result can be a reduction and/or alleviation of signs, symptoms, or causes of the disease, or any other desired changes in the biological system. For example, the “effective amount” for therapeutic use refers to the amount of the composition comprising the compound as the active ingredient of the invention that is required to significantly reduce diseases clinically. In any case, the appropriate “effective” amount can be determined using routine experiments by a person of ordinary skill in the art. Therefore, the expression “effective amount” usually refers to the amount of an active substance when it has a therapeutic effect.

The term “treat” or “treatment” used in the present application is synonymous with the term “prevent” and is intended to mean delaying the development of diseases, preventing the development of diseases and/or reducing the severity of the symptoms that will develop or are anticipated to develop. Therefore, these terms include improving existing symptoms of diseases, preventing additional symptoms, improving or preventing underlying metabolic causes of symptoms, inhibiting disorders or diseases, for example, preventing the development of disorders or diseases, alleviating disorders or diseases, degenerating disorders or diseases, alleviating conditions caused by diseases or disorders, or ceasing symptoms of diseases or disorders.

“Disease”, “disorder” and “condition” are used interchangeably herein.

Unless otherwise specified, the term “treatment” as used herein includes the effect that occurs when a subject suffers from a specific disease, disorder or condition, which reduces the severity of the disease, disorder or condition, or delays or slows down the development of the disease, disorder or condition (“therapeutic treatment”), and also includes the effect that occurs before a subject suffers from a specific disease, disorder or condition (“prophylactic treatment”).

The term “pharmaceutical”, “pharmacologically acceptable” or “pharmaceutically acceptable” is defined herein as those compounds, materials, agents, compositions and/or dosage forms that are suitable for contact with tissues of individuals (such as mammals or humans) without producing excessive toxicity, irritation of allergic reactions and other complications and with a reasonable benefit/risk ratio commensurate with them, within the scope of reasonable medical judgment.

“Administration” or “administering”, when applied to animals, humans, experimental subjects, cells, tissues, organs or biological fluids, refers to contact of exogenous drugs, therapeutic agents, diagnostic agents or compositions with subjects, cells, tissues, organs or biological fluids.

The term “co-administration” or “combined administration” as used herein is defined to encompass administration of selected therapeutic agents to a single patient, and is meant to include treatment regimens in which drugs are not necessarily administered by the same route or simultaneously.

The term “subject” as used in the present application includes mammals and non-mammals. Examples of mammals include, but are not limited to, any member of Mammalia, humans, non-human primates such as chimpanzees and other apes and monkeys; farm animals such as cows, horses, sheep, goats, pigs; domestic animals such as rabbits, dogs and cats; laboratory animals, including rodents such as rats, mice, guinea pigs, etc. Examples of non-mammals include, but are not limited to, birds, fish, etc. In one embodiment of the invention, the mammal is a human.

Preferably, the “subject” includes humans (i.e., men or women of any age group, for example, pediatric subjects (e.g., infants, children, adolescents) or adult subjects (e.g., young adults, middle-aged adults or older adults)) and/or non-human animals, for example, mammals, e.g., primates (for example, cynomolgus monkeys, rhesus monkeys), cows, pigs, horses, sheep, goats, rodents, cats and/or dogs. In some embodiments, the subject is a human. In some embodiments, the subject is a non-human animal. The terms “human”, “patient” and “subject” are used interchangeably herein.

The term “Allergin-1” is an immunosuppressive receptor belonging to the immunoglobulin-like receptor superfamily, also known as MILR1, which belongs to an immune checkpoint molecule together with programmed death molecule 1 (PD-1). Allergin-1 and PD-1 have many similar characteristics, such as expression pattern, immunoreceptor tyrosine inhibitory motif (ITIM) pattern, the ability of binding to tyrosine phosphatase SHP-1 and regulation of cell signaling by phosphorylation and dephosphorylation, etc. The activation of SHP-1 and its downstream signaling pathways plays an important role in the development of tumors and the maintenance of the properties of tumor stem cells.

MILR1 is located on the human chromosome 17. MILR1 in the invention includes wild type, mutant type or fragments thereof. A representative MILR1 gene sequence is MILR1 gene (NC_000017.11) as shown in the current international public nucleic acid database GeneBank. MILR1 gene sequence can also be shown in SEQ ID NO: 1, 2, 3, 4, 5, 6, 7, 8, 9, 10 or 11 below.
XR_002957990.1 MILR1 [organism=Homo sapiens][GeneID=284021][transcript=X4]

(SEQ ID NO: 1)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAATTCCCTTCTCCATGTTTGGACTCAAAGACTAAGGTGGTTATGAAGGGTCAAAAT
GTATCTATGTTTTGTTCCCATAAGAACAAATCACTGCAGATCACCTATTCATTGTTTCGAC
GTAAGACACACCTGGGAACCCAGGATGGAAAAGGTGAACCTGCGATTTTTAACCTAAGC
ATCACAGAAGCCCATGAATCAGGCCCCTACAAATGCAAAGCCCAAGTTACCAGCTGTTC
AAAATACAGTCGTGACTTCAGCTTCACGATTGTCGACCCGGTGACTTCCCCAGTGCTGAA
CATTATGGTCATTCAAACAGAAACAGACCGACATATAACATTACATTGCCTCTCAGTCAA
TGGCTCGCTGCCCATCAATTACACTTTCTTTGAAAACCATGTTGCCATATCACCAGCTATT
TCCAAGTATGACAGGGAGCCTGCTGAATTTAACTTAACCAAGAAGAATCCTGGAGAAGA
GGAAGAGTATAGGTGTGAAGCTAAAAACAGATTGCCTAACTATGCAACATACAGTCACC
CTGTCACCATGCCCTCAACAGGCGGAGACAGCTGTCCTTTCTGTCTGAAGCTACTACTTC
CAGGGTTATTACTGTTGCTGGTGGTGATAATCCTAATTCTGGCTTTTTGGGTACTGCCCAA
ATACAAAACAAGAAAAGCTATGAGAAATAATGTGCCCAGGGACCGTGGAGACACAGCC
ATGGAAGTTGGAATCTATGCAAATATCCTTGAAAAACAAGCAAAGGAGGAATCTGTGCC
AGAAGTGGGATCCAGGCCGTGTGTTTCCACAGCCCAAGATGAGGCCAAACACTCCCAGG
AGCTACAGTATGCCACCCCCGTGTTCCAGGAGGTGGCACCAAGAGAGCAAGAAGCCTGT
GATTCTTATAAATCTGGATATGTCTATTCTGAACTCAACTTCTGAAATTTACAGAAACAA
ACTACATCTCAGGTGAGTACTAAAACTTGATCATCAATTACAGAGCCATTTTGACTAATA
TGGAAAGTTCATGGAGGGCCAGTTGCAATCCCCAAGATCCAGCAAGACTGCCTCGCCTTT
CCACCCGACACCTGTTTTGAAGCATGAAATCGTGAAGCATACCGTGAAGAAGGTTCTCCC
GTAGTTTCCCAGAAGTTTTTAATACGTTCTCAAGAAATGCTACTA.

XR_002957989.1 MILR1 [organism=Homo sapiens][GeneID=284021][transcript=X2]

(SEQ ID NO: 2)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAATTCCCTTCTCCATGTTTGGACTCAAAGACTAAGGTGGTTATGAAGGGTCAAAAT
GTATCTATGTTTTGTTCCCATAAGAACAAATCACTGCAGATCACCTATTCATTGTTTCGAC
GTAAGACACACCTGGGAACCCAGGATGGAAAAGGTGAACCTGCGATTTTTAACCTAAGC
ATCACAGAAGCCCATGAATCAGGCCCCTACAAATGCAAAGCCCAAGTTACCAGCTGTTC
AAAATACAGTCGTGACTTCAGCTTCACGATTGTCGACCCGGTGACTTCCCCAGTGCTGAA
CATTATGGTCATTCAAACAGAAACAGACCGACATATAACATTACATTGCCTCTCAGTCAA
TGGCTCGCTGCCCATCAATTACACTTTCTTTGAAAACCATGTTGCCATATCACCAGCTATT
TCCAAGTATGACAGGGAGCCTGCTGAATTTAACTTAACCAAGAAGAATCCTGGAGAAGA
GGAAGAGTATAGGTGTGAAGCTAAAAACAGATTGCCTAACTATGCAACATACAGTCACC
CTGTCACCATGCCCTCAACAGGCGGAGACAGCTGTCCTTTCTGTCTGAAGCTACTACTTC
CAGGGTTATTACTGTTGCTGGTGGTGATAATCCTAATTCTGGCTTTTTGGGTACTGCCCAA
ATACAAAACAAGAAAAGCTATGAGAAATAATGTGCCCAGGGACCGTGGAGACACAGCC
ATGGAAGTTGGAATCTATGCAAATATCCTTGAAAAACAAGCAAAGGAGGAATCTGTGCC
AGAAGTGGGATCCAGGCCGTGTGTTTCCACAGCCCAAGATGAGGCCAAACACTCCCAGG
AGCTACAGTATGCCACCCCCGTGTTCCAGGAGGTGGCACCAAGAGAGCAAGAAGCCTGT
GATTCTTATAAATCTGGATATGTCTATTCTGAACTCAACTTCTGAAATTTACAGAAACAA
ACTACATCTCAGGTGAGTACTAAAACTTGATCATCAATTACAGAGCCATTTTGACTAATA
TGGAAAGTTCATGGAGATGTGGCCAGTGACAACCCTCCTCAAGCCAGCATCTGTGACCTT
TTTATATGACCCATTAGTCTTCAAGCAATTCTTTGCTTTTTAGCACCATAAAGTGTTTCAA
ACTCACCTTGTACTTTCCCTAACCCCAGACCCGGAATCAGCCATTTCTCCCAGTACTGGTT
CATCTTAGAAACCAAGATCTGGAGGCCGGGCGCAGTGGC.

XM_024450708.1 MILR1 [organism=Homo sapiens][GeneID=284021][transcript=X3]

(SEQ ID NO: 3)
CACAGAGGAAGACAGATTGGTCGAACAAACCAGTATTATGCAAACCTCATCCAAACCCT
CTGATTTCCTTAACTTGGCTAAGAAAAAGAGGAAGTTCTCCGAGTTACTCACCACTGTGG
TTCTACTATGCCTTCTGACCCCGTCTTGGACTTCAACTGGGAGAATGTGGAGCCATTTGA
ACAGGCTCCTCTTCTGGAGCATATTTTCTTCTGTCACTTGTAGAAAAGCTGTATTGGATTG
TGAGGCAATGAAAACAAATGAATTCCCTTCTCCATGTTTGGACTCAAAGACTAAGGTGGT
TATGAAGGGTCAAAATGTATCTATGTTTTGTTCCCATAAGAACAAATCACTGCAGATCAC
CTATTCATTGTTTCGACGTAAGACACACCTGGGAACCCAGGATGGAAAAGGTGAACCTG
CGATTTTTAACCTAAGCATCACAGAAGCCCATGAATCAGGCCCCTACAAATGCAAAGCCC
AAGTTACCAGCTGTTCAAAATACAGTCGTGACTTCAGCTTCACGATTGTCGACCCGGTGA
CTTCCCCAGTGCTGAACATTATGGTCATTCAAACAGAAACAGACCGACATATAACATTAC
ATTGCCTCTCAGTCAATGGCTCGCTGCCCATCAATTACACTTTCTTTGAAAACCATGTTGC
CATATCACCAGCTATTTCCAAGTATGACAGGGAGCCTGCTGAATTTAACTTAACCAAGAA
GAATCCTGGAGAAGAGGAAGAGTATAGGTGTGAAGCTAAAAACAGATTGCCTAACTATG
CAACATACAGTCACCCTGTCACCATGCCCTCAACAGGCGGAGACAGCTGTCCTTTCTGTC
TGAAGCTACTACTTCCAGGGTTATTACTGTTGCTGGTGGTGATAATCCTAATTCTGGCTTT
TTGGGTACTGCCCAAATACAAAACAAGAAAAGCTATGAGAAATAATGTGCCCAGGGACC
GTGGAGACACAGCCATGGAAGTTGGAATCTATGCAAATATCCTTGAAAAACAAGCAAAG
GAGGAATCTGTGCCAGAAGTGGGATCCAGGCCGTGTGTTTCCACAGCCCAAGATGAGGC
CAAACACTCCCAGGAGCTACAGTATGCCACCCCCGTGTTCCAGGAGGTGGCACCAAGAG
AGCAAGAAGCCTGTGATTCTTATAAATCTGGATATGTCTATTCTGAACTCAACTTCTGAA
ATTTACAGAAACAAACTACATCTCAGGTGAGTACTAAAACTTGATCATCAATTACAGAGC
CATTTTGACTAATATGGAAAGTTCATGGAGGTAGGTAGCAAAAGCTGAAGCTGTGACAA
TGGTCAAGGGTCAAACTCTACATCAGCCAGGAATGCTAGGTGAGGGCTTTGAATTTTACC
TTACACAGAAAGGCTAATTAACAAACACATCTGAGCCCAACAAATGTTTTTACCAAAGTA
ACTTTGTAATCTAGAACAATGAAAATTGTTACAAAGAGTTTTTGTATTTGTATAATATATT
AATAGCTACATACATCAAATATAATTTCTTGTATGTCAGAAGAATTACCACTGACACATT
AAGACAATTTATGTATTAACTATTAAATTAAAGGTAATTATTGCAGTACCTTTCTCCACCA
CTGGAGTCGATGACGTAACCAGAAATCAAGCCACTGGTTTGAAGTTCTCGGAGGAGTAA
ACCATACTAACGAAGCTTCAGTCTTCTCACCAA.

XM_024450708.1 MILR1 [organism=Homo sapiens][GeneID=284021][transcript=X6]

(SEQ ID NO: 4)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAATTCCCTTCTCCATGTTTGGACTCAAAGACTAAGGTGGTTATGAAGGGTCAAAAT
GTATCTATGTTTTGTTCCCATAAGAACAAATCACTGCAGATCACCTATTCATTGTTTCGAC
GTAAGACACACCTGGGAACCCAGGATGGAAAAGGTGAACCTGCGATTTTTAACCTAAGC
ATCACAGAAGCCCATGAATCAGGCCCCTACAAATGCAAAGCCCAAGTTACCAGCTGTTC
AAAATACAGTCGTGACTTCAGCTTCACGATTGTCGACCCGGTGACTTCCCCAGTGCTGAA
CATTATGGTCATTCAAACAGAAACAGACCGACATATAACATTACATTGCCTCTCAGTCAA
TGGCTCGCTGCCCATCAATTACACTTTCTTTGAAAACCATGTTGCCATATCACCAGCTATT
TCCAAGTATGACAGGGAGCCTGCTGAATTTAACTTAACCAAGAAGAATCCTGGAGAAGA
GGAAGAGTATAGGTGTGAAGCTAAAAACAGATTGCCTAACTATGCAACATACAGTCACC
CTGTCACCATGCCCTCAACAGGCGGAGACAGCTGTCCTTTCTGTCTGAAGCTACTACTTC
CAGGGTTATTACTGTTGCTGGTGGTGATAATCCTAATTCTGGCTTTTTGGGTACTGCCCAA
ATACAAAACAAGAAAAGCTATGAGAAATAATGTGCCCAGGGACCGTGGAGACACAGCC
ATGGAAGTTGGAATCTATGCAAATATCCTTGAAAAACAAGCAAAGGAGGAATCTGTGCC
AGAAGTGGGATCCAGGCCGTGTGTTTCCACAGCCCAAGATGAGGCCAAACACTCCCAGG
AGCTACAGTATGCCACCCCCGTGTTCCAGGAGGTGGCACCAAGAGAGCAAGAAGCCTGT
GATTCTTATAAATCTGGATATGTCTATTCTGAACTCAACTTCTGAAATTTACAGAAACAA
ACTACATCTCAGGGGCCAGTTGCAATCCCCAAGATCCAGCAAGACTGCCTCGCCTTTCCA
CCCGACACCTGTTTTGAAGCATGAAATCGTGAAGCATACCGTGAAGAAGGTTCTCCCGTA
GTTTCCCAGAAGTTTTTAATACGTTCTCAAGAAATGCTACTA.

XM_024450709.1 MILR1 [organism=Homo sapiens][GeneID=284021][transcript=X7]

(SEQ ID NO: 5)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAATTCCCTTCTCCATGTTTGGACTCAAAGACTAAGGTGGTTATGAAGGGTCAAAAT
GTATCTATGTTTTGTTCCCATAAGAACAAATCACTGCAGATCACCTATTCATTGTTTCGAC
GTAAGACACACCTGGGAACCCAGGATGGAAAAGGTGAACCTGCGATTTTTAACCTAAGC
ATCACAGAAGCCCATGAATCAGGCCCCTACAAATGCAAAGCCCAAGTTACCAGCTGTTC
AAAATACAGTCGTGACTTCAGCTTCACGATTGTCGACCCGGTGACTTCCCCAGTGCTGAA
CATTATGGTCATTCAAACAGAAACAGACCGACATATAACATTACATTGCCTCTCAGTCAA
TGGCTCGCTGCCCATCAATTACACTTTCTTTGAAAACCATGTTGCCATATCACCAGCTATT
TCCAAGTATGACAGGGAGCCTGCTGAATTTAACTTAACCAAGAAGAATCCTGGAGAAGA
GGAAGAGTATAGGTGTGAAGCTAAAAACAGATTGCCTAACTATGCAACATACAGTCACC
CTGTCACCATGCCCTCAACAGGCGGAGACAGCTGTCCTTTCTGTCTGAAGCTACTACTTC
CAGGGTTATTACTGTTGCTGGTGGTGATAATCCTAATTCTGGCTTTTTGGGTACTGCCCAA
ATACAAAACAAGAAAAGCTATGAGAAATAATGTGCCCAGGGACCGTGGAGACACAGCC
ATGGAAGTTGGAATCTATGCAAATATCCTTGAAAAACAAGCAAAGGAGGAATCTGTGCC
AGAAGTGGGATCCAGGCCGTGTGTTTCCACAGCCCAAGATGAGGCCAAACACTCCCAGG
AGCTACAGTATGCCACCCCCGTGTTCCAGGAGGTGGCACCAAGAGAGCAAGAAGCCTGT
GATTCTTATAAATCTGGATATGTCTATTCTGAACTCAACTTCTGAAATTTACAGAAACAA
ACTACATCTCAGGTAGAGACGGGGTTTTGC.

XM_024450707.1 MILR1 [organism=Homo sapiens][GeneID=284021][transcript=X5]

(SEQ ID NO: 6)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAATTCCCTTCTCCATGTTTGGACTCAAAGACTAAGGTGGTTATGAAGGGTCAAAAT
GTATCTATGTTTTGTTCCCATAAGAACAAATCACTGCAGATCACCTATTCATTGTTTCGAC
GTAAGACACACCTGGGAACCCAGGATGGAAAAGGTGAACCTGCGATTTTTAACCTAAGC
ATCACAGAAGCCCATGAATCAGGCCCCTACAAATGCAAAGCCCAAGTTACCAGCTGTTC
AAAATACAGTCGTGACTTCAGCTTCACGATTGTCGACCCGGTGACTTCCCCAGTGCTGAA
CATTATGGTCATTCAAACAGAAACAGACCGACATATAACATTACATTGCCTCTCAGTCAA
TGGCTCGCTGCCCATCAATTACACTTTCTTTGAAAACCATGTTGCCATATCACCAGCTATT
TCCAAGTATGACAGGGAGCCTGCTGAATTTAACTTAACCAAGAAGAATCCTGGAGAAGA
GGAAGAGTATAGGTGTGAAGCTAAAAACAGATTGCCTAACTATGCAACATACAGTCACC
CTGTCACCATGCCCTCAACAGGCGGAGACAGCTGTCCTTTCTGTCTGAAGCTACTACTTC
CAGGGTTATTACTGTTGCTGGTGGTGATAATCCTAATTCTGGCTTTTTGGGTACTGCCCAA
ATACAAAACAAGAAAAGCTATGAGAAATAATGTGCCCAGGGACCGTGGAGACACAGCC
ATGGAAGTTGGAATCTATGCAAATATCCTTGAAAAACAAGCAAAGGAGGAATCTGTGCC
AGAAGTGGGATCCAGGCCGTGTGTTTCCACAGCCCAAGATGAGGCCAAACACTCCCAGG
AGCTACAGTATGCCACCCCCGTGTTCCAGGAGGTGGCACCAAGAGAGCAAGAAGCCTGT
GATTCTTATAAATCTGGATATGTCTATTCTGAACTCAACTTCTGAAATTTACAGAAACAA
ACTACATCTCAGGTAGAGACAGGGTTTTGCCATGTTGGCCAGGCTGGTCTTCAACTCCTG
ACCTCAAGTGATCCGCCCACCTCGGACTCCCAAAGTGCTGGGATTACAGGCGTGAGCCAC
CTCGCCTGGCCCTCCATTTCCTGATCTAGTCTTATATCCACGCTCACCACCTCAGCACGCT
CAGACCCACGCTGCTGTGGGCTCCTCTGGCTCCTGGAAGAGTGCGTCCGCAGATGCTGCA
GTCTTTGTGTGGCTCAGCAATTGCCACTCACATCAGGAACTGCCTTTACCCTGTCAGGCTC
TACTGAGACCCGACCCTGGTTATTAAGCTATAGGGGAGACAAGGATGGATCTTAAAGAA
GACAAGCAAAATGTAGTGAAGCAAATAGAATGGTGGTTCCTGGGGGATGGGGGCAGGG
GACAACGAGGAGTGACTGGCTAACAGATACAGCGTTTCAGTTTGGAAAGACAAAAAAGT
TCTGGAAAAAAGATGGAAGGTGGTGATGGTTGCACAATAATATGAGTTTTGTTGTTGTTG
TTTTTTGAG.

XM_017024487.1 MILR1 [organism=Homo sapiens][GeneID=284021][transcript=X8]

(SEQ ID NO: 7)
TTGGTCGAACAAACCAGTATTATGCAAACCTCATCCAAACCCTCTGATTTCCTTAACTTG
GCTAAGAAAAAGAGGAAGTTCTCCGAGTTACTCACCACTGTGGTTCTACTATGCCTTCTG
ACCCCGTCTTGGACTTCAACTGGGAGAATGTGGAGCCATTTGAACAGGCTCCTCTTCTGG
AGCATATTTTCTTCTGTCACTTGTAGAAAAGCTGTATTGGATTGTGAGGCAATGAAAACA
AATGAACAAATCACTGCAGATCACCTATTCATTGTTTCGACGTAAGACACACCTGGGAAC
CCAGGATGGAAAAGGTGAACCTGCGATTTTTAACCTAAGCATCACAGAAGCCCATGAAT
CAGGCCCCTACAAATGCAAAGCCCAAGTTACCAGCTGTTCAAAATACAGTCGTGACTTCA
GCTTCACGATTGTCGACCCGGTGACTTCCCCAGTGCTGAACATTATGGTCATTCAAACAG
AAACAGACCGACATATAACATTACATTGCCTCTCAGTCAATGGCTCGCTGCCCATCAATT
ACACTTTCTTTGAAAACCATGTTGCCATATCACCAGCTATTTCCAAGTATGACAGGGAGC
CTGCTGAATTTAACTTAACCAAGAAGAATCCTGGAGAAGAGGAAGAGTATAGGTGTGAA
GCTAAAAACAGATTGCCTAACTATGCAACATACAGTCACCCTGTCACCATGCCCTCAACA
GGCGGAGACAGCTGTCCTTTCTGTCTGAAGCTACTACTTCCAGGGTTATTACTGTTGCTGG
TGGTGATAATCCTAATTCTGGCTTTTTGGGTACTGCCCAAATACAAAACAAGAAAAGCTA
TGAGAAATAATGTGCCCAGGGACCGTGGAGACACAGCCATGGAAGTTGGAATCTATGCA
AATATCCTTGAAAAACAAGCAAAGGAGGAATCTGTGCCAGAAGTGGGATCCAGGCCGTG
TGTTTCCACAGCCCAAGATGAGGCCAAACACTCCCAGGAGCTACAGTATGCCACCCCCGT
GTTCCAGGAGGTGGCACCAAGAGAGCAAGAAGCCTGTGATTCTTATAAATCTGGATATG
TCTATTCTGAACTCAACTTCTGAAATTTACAGAAACAAACTACATCTCAGGGTAAGATGC
TTTTTATGAAGCTGATTTCCATGAACAAAAAGCAAACTTGAGGCTGAGGCAGGTGGATTA
CTTGAGGTCAGGAGTTCGAGACCAGCCTGGCCAACATGGCAAAAACCCCAT.

NM_001369493.1 MILR1 [organism=Homo sapiens][GeneID=284021][transcript=4]

(SEQ ID NO: 8)
ACCACTGTGGTTCTACTATGCCTTCTGACCCCGTCTTGGACTTCAACTGGGAGAATGTGG
AGCCATTTGAACAGGCTCCTCTTCTGGAGCATATTTTCTTCTGTCACTTGTAGAAAAGCTG
TATTGGATTGTGAGGCAATGAAAACAAATGAATTCCCTTCTCCATGTTTGGACTCAAAGA
CTAAGGTGGTTATGAAGGGTCAAAATGTATCTATGTTTTGTTCCCATAAGAACAAATCAC
TGCAGATCACCTATTCATTGTTTCGACGTAAGACACACCTGGGAACCCAGGATGGAAAA
GGTGAACCTGCGATTTTTAACCTAAGCATCACAGAAGCCCATGAATCAGGCCCCTACAAA
TGCAAAGCCCAAGTTACCAGCTGTTCAAAATACAGTCGTGACTTCAGCTTCACGATTGTC
GACCCGGTGACTTCCCCAGTGCTGAACATTATGGTCATTCAAACAGAAACAGACCGACAT
ATAACATTACATTGCCTCTCAGTCAATGGCTCGCTGCCCATCAATTACACTTTCTTTGAAA
ACCATGTTGCCATATCACCAGCTATTTCCAAGTATGACAGGGAGCCTGCTGAATTTAACT
TAACCAAGAAGAATCCTGGAGAAGAGGAAGAGTATAGGTGTGAAGCTAAAAACAGATT
GCCTAACTATGCAACATACAGTCACCCTGTCACCATGCCCTCAACAGGCGGAGACAGCTG
TCCTTTCTGTCTGAAGCTACTACTTCCAGGGTTATTACTGTTGCTGGTGGTGATAATCCTA
ATTCTGGCTTTTTGGGTACTGCCCAAATACAAAACAAGAAAAGCTATGAGAAATAATGTG
CCCAGGGACCGTGGAGACACAGCCATGGAAGTTGGAATCTATGCAAATATCCTTGAAAA
ACAAGCAAAGGAGGAATCTGTGCCAGAAGTGGGATCCAGGCCGTGTGTTTCCACAGCCC
AAGATGAGGCCAAACACTCCCAGGAGCTACAGTATGCCACCCCCGTGTTCCAGGAGGTG
GCACCAAGAGAGCAAGAAGCCTGTGATTCTTATAAATCTGGATATGTCTATTCTGAACTC
AACTTCTGAAATTTACAGAAACAAACTACATCTCAGGGATTTAAAGGGGAGACCTGGCC
AGAGGAATCATCAACAGTGGATTTGTGATGACTGGAGTTCATTTCCTTCTTTCTCAACTGT
CCTGGAAGGAAATGGACTAGTGCCTTACCTCTGACTCTTCCGCAGCACACACCCACCACC
AGAGACAGTAACTGAGATCTGCAGTAGCACAGAAACTTACAGGTTAGAAAAATTAGACA
CCTAAGGATTTCATGACTCCAGACCTGTTTCCTTAGCATAATACCACACCAGTAGGAGAA
ATACAACTTACGAAATTTCTTTGTAAATAAATTCTATAACATTTTGGGTAAAAATTTATCA
TAAAAAAATTATTAAAAGCAATTGGGTTGGAAGGAAGTTCTGTCAAAATACAATCCAGG
TGTGCAGTTCTCTGATAAATTTAGCAGTGGTTACTACTGAGGCAGTCTCAAAAAAAAGTG
TAAAAGGACTTTAAGGGGTGATGAAAATTTTCTACATTGCCTTTGTGGAGATGGTTATAC
ATTTGTCAATACTCATCAATTTGTACATTTAAAAGAGGTAAATTTATTGTATGTAAATTGT
ATCTTAATAAAACTGATTAAAACAGACATACAAACCCCA.

NM_001085423.2 MILR1 [organism=Homo sapiens][GeneID=284021][transcript=L]

(SEQ ID NO: 9)
ACCACTGTGGTTCTACTATGCCTTCTGACCCCGTCTTGGACTTCAACTGGGAGAATGTGG
AGCCATTTGAACAGGCTCCTCTTCTGGAGCATATTTTCTTCTGTCACTTGTAGAAAAGCTG
TATTGGATTGTGAGGCAATGAAAACAAATGAATTCCCTTCTCCATGTTTGGACTCAAAGA
CTAAGGTGGTTATGAAGGGTCAAAATGTATCTATGTTTTGTTCCCATAAGAACAAATCAC
TGCAGATCACCTATTCATTGTTTCGACGTAAGACACACCTGGGAACCCAGGATGGAAAA
GGTGAACCTGCGATTTTTAACCTAAGCATCACAGAAGCCCATGAATCAGGCCCCTACAAA
TGCAAAGCCCAAGTTACCAGCTGTTCAAAATACAGTCGTGACTTCAGCTTCACGATTGTC
GACCCGGTGACTTCCCCAGTGCTGAACATTATGGTCATTCAAACAGAAACAGACCGACAT
ATAACATTACATTGCCTCTCAGTCAATGGCTCGCTGCCCATCAATTACACTTTCTTTGAAA
ACCATGTTGCCATATCACCAGCTATTTCCAAGTATGACAGGGAGCCTGCTGAATTTAACT
TAACCAAGAAGAATCCTGGAGAAGAGGAAGAGTATAGGTGTGAAGCTAAAAACAGATT
GCCTAACTATGCAACATACAGTCACCCTGTCACCATGCCCTCAACAGGCGGAGACAGCTG
TCCTTTCTGTCTGAAGCTACTACTTCCAGGGTTATTACTGTTGCTGGTGGTGATAATCCTA
ATTCTGGCTTTTTGGGTACTGCCCAAATACAAAACAAGAAAAGCTATGAGAAATAATGTG
CCCAGGGACCGTGGAGACACAGCCATGGAAGTTGGAATCTATGCAAATATCCTTGAAAA
ACAAGCAAAGGAGGAATCTGTGCCAGAAGTGGGATCCAGGCCGTGTGTTTCCACAGCCC
AAGATGAGGCCAAACACTCCCAGGAGCTACAGTATGCCACCCCCGTGTTCCAGGAGGTG
GCACCAAGAGAGCAAGAAGCCTGTGATTCTTATAAATCTGGATATGTCTATTCTGAACTC
AACTTCTGAAATTTACAGAAACAAACTACATCTCAGGATGGAGTCTCACTCTGTTGCCCA
GGCTGGAGTTCAGTAGCGCGATCTTGGCTCACTTCAATCTCCATCTTCCCAGTTCAAGCG
ATTCTCATGCCTCGACCTCCCGAGTAGCTGGGATTACAGGTGCCCGCTACCACGCCCAGC
TAATTTTTGTATTTTTAGTAGAGATGGGGTTTCACTATGGTGGCCAGGCTGGTCTTGAACT
CCTGACCTCAGATGATCTGCCTGCCTCGGCCTCCCAAAGTGCTGGAACTACAAGCCTGAG
CCACCGTGCCCGGCCCTGAATCGCTTTAGTAAATAAAGGGTCTCCAAGAATAAATTCATC
CGAACATGCA.

NM_001291316.2 MILR1 [organism=Homo sapiens][GeneID=284021][transcript=S1]

(SEQ ID NO: 10)
ACCACTGTGGTTCTACTATGCCTTCTGACCCCGTCTTGGACTTCAACTGGGAGAATGTGG
AGCCATTTGAACAGGCTCCTCTTCTGGAGCATATTTTCTTCTGTCACTTGTAGAAAAGCTG
TATTGGATTGTGAGGCAATGAAAACAAATGAATTCCCTTCTCCATGTTTGGACTCAAAGA
CTAAGGTGGTTATGAAGGGTCAAAATGTATCTATGTTTTGTTCCCATAAGAACAAATCAC
TGCAGATCACCTATTCATTGTTTCGACGTAAGACACACCTGGGAACCCAGGATGGAAAA
GGTGAACCTGCGATTTTTAACCTAAGCATCACAGAAGCCCATGAATCAGGCCCCTACAAA
TGCAAAGCCCAAGTTACCAGCTGTTCAAAATACAGTCGTGACTTCAGCTTCACGATTGTC
GGCGGAGACAGCTGTCCTTTCTGTCTGAAGCTACTACTTCCAGGGTTATTACTGTTGCTGG
TGGTGATAATCCTAATTCTGGCTTTTTGGGTACTGCCCAAATACAAAACAAGAAAAGCTA
TGAGAAATAATGTGCCCAGGGACCGTGGAGACACAGCCATGGAAGTTGGAATCTATGCA
AATATCCTTGAAAAACAAGCAAAGGAGGAATCTGTGCCAGAAGTGGGATCCAGGCCGTG
TGTTTCCACAGCCCAAGATGAGGCCAAACACTCCCAGGAGCTACAGTATGCCACCCCCGT
GTTCCAGGAGGTGGCACCAAGAGAGCAAGAAGCCTGTGATTCTTATAAATCTGGATATG
TCTATTCTGAACTCAACTTCTGAAATTTACAGAAACAAACTACATCTCAGGATGGAGTCT
CACTCTGTTGCCCAGGCTGGAGTTCAGTAGCGCGATCTTGGCTCACTTCAATCTCCATCTT
CCCAGTTCAAGCGATTCTCATGCCTCGACCTCCCGAGTAGCTGGGATTACAGGTGCCCGC
TACCACGCCCAGCTAATTTTTGTATTTTTAGTAGAGATGGGGTTTCACTATGGTGGCCAG
GCTGGTCTTGAACTCCTGACCTCAGATGATCTGCCTGCCTCGGCCTCCCAAAGTGCTGGA
ACTACAAGCCTGAGCCACCGTGCCCGGCCCTGAATCGCTTTAGTAAATAAAGGGTCTCCA
AGAATAAATTCATCCGAACATGCA.

NM_001291317.2 MILR1 [organism=Homo sapiens][GeneID=284021][transcript=S2]

(SEQ ID NO: 11)
ACCACTGTGGTTCTACTATGCCTTCTGACCCCGTCTTGGACTTCAACTGGGAGAATGTGG
AGCCATTTGAACAGGCTCCTCTTCTGGAGCATATTTTCTTCTGTCACTTGTAGAAAAGCTG
TATTGGATTGTGAGGCAATGAAAACAAATGACCCGGTGACTTCCCCAGTGCTGAACATTA
TGGTCATTCAAACAGAAACAGACCGACATATAACATTACATTGCCTCTCAGTCAATGGCT
CGCTGCCCATCAATTACACTTTCTTTGAAAACCATGTTGCCATATCACCAGCTATTTCCAA
GTATGACAGGGAGCCTGCTGAATTTAACTTAACCAAGAAGAATCCTGGAGAAGAGGAAG
AGTATAGGTGTGAAGCTAAAAACAGATTGCCTAACTATGCAACATACAGTCACCCTGTCA
CCATGCCCTCAACAGGCGGAGACAGCTGTCCTTTCTGTCTGAAGCTACTACTTCCAGGGT
TATTACTGTTGCTGGTGGTGATAATCCTAATTCTGGCTTTTTGGGTACTGCCCAAATACAA
AACAAGAAAAGCTATGAGAAATAATGTGCCCAGGGACCGTGGAGACACAGCCATGGAA
GTTGGAATCTATGCAAATATCCTTGAAAAACAAGCAAAGGAGGAATCTGTGCCAGAAGT
GGGATCCAGGCCGTGTGTTTCCACAGCCCAAGATGAGGCCAAACACTCCCAGGAGCTAC
AGTATGCCACCCCCGTGTTCCAGGAGGTGGCACCAAGAGAGCAAGAAGCCTGTGATTCT
TATAAATCTGGATATGTCTATTCTGAACTCAACTTCTGAAATTTACAGAAACAAACTACA
TCTCAGGATGGAGTCTCACTCTGTTGCCCAGGCTGGAGTTCAGTAGCGCGATCTTGGCTC
ACTTCAATCTCCATCTTCCCAGTTCAAGCGATTCTCATGCCTCGACCTCCCGAGTAGCTGG
GATTACAGGTGCCCGCTACCACGCCCAGCTAATTTTTGTATTTTTAGTAGAGATGGGGTTT
CACTATGGTGGCCAGGCTGGTCTTGAACTCCTGACCTCAGATGATCTGCCTGCCTCGGCC
TCCCAAAGTGCTGGAACTACAAGCCTGAGCCACCGTGCCCGGCCCTGAATCGCTTTAGTA
AATAAAGGGTCTCCAAGAATAAATTCATCCGAACATGCA.

The term “AML” subtype: In 1976, the acute leukemia classification standard proposed by the French, American, and British (FAB) Leukemia Classification Collaborative Group has been used as the basic method for diagnosis and classification of diseases, and has been supplemented and modified many times later. The FAB classification of AML is the most commonly used type currently. AML can be divided into 8 types, namely M0 (acute myeloblastic leukemia, minimally differentiated), M1 (acute myeloblastic leukemia, undifferentiated), M2 (acute myeloblastic leukemia, partially differentiated), M3 (acute promyelocytic leukemia), M4 (acute myelomonocytic leukemia), M5 (acute monocytic leukemia), M6 (erythroleukemia), and M7 (acute megakaryocytic leukemia), respectively.

Monocytic AML cells: mononuclear cells isolated from bone marrow of AML patients are monocytic AML cells. For example, the bone marrow fluid of AML patients can be extracted by bone marrow puncture using Ficoll-Paque separation solution for separation of monocytes.

Normal monocytes: monocytes in blood of a normal healthy body are normal monocytes. For example, they can be separated from cord blood of healthy full-term newborns using Ficoll-Paque separation solution.

Leukemia stem cells (LSCs), also known as leukemia initiating cells (LICs), are a group of leukemia cells that have ability to self-renew and produce heterogeneous leukemia cell populations. In 1994, Tsvee Lapidot et al. firstly isolated CD34⁺CD38⁻ LICs subpopulation from bone marrow cells of AML patients, and found that this subpopulation has powerful self-renewal ability like HSCs and can rebuild leukemia in immunodeficient mice. In 2007, Fumihiko Ishikawa et al. found that LSCs can escape the killing effect of chemical drugs, leading to drug resistance and easy recurrence.

The term “inhibitor” refers to a modulator capable of inhibiting or down-regulating the expression or activity of substances. For example, it can be a siRNA capable of specifically knocking down or silencing Allergin-1 or an antibody capable of specifically binding and inhibiting the activity of Allergin-1.

The term “antagonist” refers to a modulator capable of antagonizing the activity of Allergin-1 and reducing or inhibiting the interaction between Allergin-1 and Allergin-1 ligand. For example, it can be a competitive binding agent for Allergin-1 ligand.

An “antibody” is an immunoglobulin molecule capable of specifically binding to a target such as carbohydrates, polynucleotides, lipids, and polypeptides via at least one antigen recognition site located in a variable region of the immunoglobulin molecule. As used herein, the term encompasses not only intact polyclonal or monoclonal antibodies, but also fragments thereof (such as Fab, Fab′, F(ab′)2, Fv), single chain variable fragment (scFv) and domain antibodies (including such as shark and camelid antibodies) and fusion proteins containing antibodies, and any other modified configurations of immunoglobulin molecules containing antigen recognition sites. The antibody includes any class of antibody, such as IgG, IgA, or IgM (or subclasses thereof), and the antibody is not necessary to be of any specific class. According to amino acid sequence of heavy chain constant region of the antibody, immunoglobulins can be classified into different classes. There are five main classes of immunoglobulins: IgA, IgD, IgE, IgG, and IgM, and several of these can be further divided into subclasses (isotypes), such as IgG1, IgG2, IgG3, IgG4, IgA1, and IgA2. The heavy chain constant regions corresponding to different types of immunoglobulins are called α, δ, ε, γ, and μ, respectively. Subunit structures and three-dimensional configurations of various classes of immunoglobulins are well known.

As used herein, the term “antibody” includes intact antibodies and any antigen-binding fragments (i.e., “antigen-binding portions”) or single chains thereof. “Antibody” refers to a protein comprising at least two heavy chains (H) and two light chains (L) connected to each other via disulfide bonds, or an antigen-binding portion thereof. Each heavy chain consists of a heavy chain variable region (abbreviated as VH herein) and a heavy chain constant region. The heavy chain constant region consists of three domains, CH1, CH2 and CH3. Each light chain consists of a light chain variable region (abbreviated as VL herein) and a light chain constant region. The light chain constant region consists of one domain, CL. VH and VL regions can be further subdivided into hypervariable regions, called complementary determining regions (CDRs), interspersed with more conservative regions called framework regions (FRs). Each VH and VL consists of three CDRs and four FRs, arranged in the following order from amino terminal to carboxyl terminal: FR1, CDR1, FR2, CDR2, FR3, CDR3, FR4. Variable regions of heavy and light chains contain binding domains that interact with antigens.

The term “antibody” as used in the application refers to an immunoglobulin or fragments or derivatives thereof, and includes any polypeptide containing antigen binding sites thereof, regardless of whether it is produced in vitro or in vivo. The term includes, but is not limited to, polyclonal, monoclonal, monospecific, multispecific, non-specific, humanized, single-chain, chimeric, synthetic, recombinant, hybrid, mutant, grafted antibodies. The term “antibody” also includes antibody fragments such as Fab, F(ab′)2, FV, scFv, Fd, dAb, and other antibody fragments that retain antigen-binding function, that is, are capable of specifically binding to PD-1. Normally, such fragments will include antigen-binding fragments.

The term “antigen-binding fragment” or “antigen-binding portion” of an antibody as used herein refers to one or more fragments of an intact antibody that retain the ability to specifically bind to a given antigen. The antigen-binding function of an antibody can be performed by fragments of intact antibodies. Examples of binding fragments encompassed in the term “antigen-binding fragment” of an antibody include Fab; Fab′; F (ab′)2; Fd fragment consisting of VH domain and CH1 domain; Fv fragment consisting of VL domain and VH domain of single arm of an antibody; single domain antibody (dAb) fragment (Ward et al., Nature 341:544-546, 1989); and separated complementary determining region (CDR). The terms “antigen-binding fragment”, “antigen-binding domain” and “binding fragment” refer to an antibody molecule that contains amino acids responsible for the binding between a specific antibody and an antigen. For example, the antigen is large, and the antigen-binding fragment only binds to a portion of the antigen. That is, the portion of the antigen molecule responsible for the specific interaction with the antigen-binding fragment is called “epitope” or “antigenic determinant”.

The antigen-binding fragment usually comprises an antibody light chain variable region (VL) and an antibody heavy chain variable region (VH). However, it does not necessarily comprise the both. For example, a so-called Fd antibody fragment consists only of VH domain, but still retains some of the antigen-binding function of intact antibody.

The term “homology” refers to sequence similarity between two polypeptide sequences when they are optimally aligned. When the position in both of the two compared sequences is occupied by the same amino acid monomer subunit, for example, if the position in light chain CDRs of two different Abs is occupied by alanine, the two Abs are homologous at that position. The percent homology is the number of homology position shared by two sequences divided by the total number of positions compared×100. For example, if 8 out of 10 positions in two sequences are matched or homologous when the sequences are optimally aligned, the two sequences are 80% homologous. Generally, the comparison is performed when two sequences are aligned to produce the maximum percent homology. For example, the comparison can be performed by the BLAST algorithm, where parameters of the algorithm are selected to produce the largest match between each sequence over the full length of each reference sequence.

In the invention, the terms “chip”, “microarray” and “array” can be equivalently replaced, including but not limited to: DNA microarray (for example, cDNA microarray and oligonucleotide microarray), protein microarray, tissue microarray, transfection or cell microarray, chemical compound microarray and antibody microarray. The DNA microarray, commonly referred to as gene chip, DNA chip, or biochip, is collection of microscopic DNA dots that are connected onto a solid surface (for example, glass, plastic, or silicon chips) to form an array for simultaneous expression profiling or expression level monitoring of thousands of genes. The fixed DNA fragments are referred to as probes, thousands of which can be used in a single DNA microarray. Microarrays can be used to identify disease genes or transcripts (e.g., ncRNA) by comparing gene expression in disease and normal cells. Microarrays can be manufactured using a variety of techniques, including but not limited to: printing onto glass slides with fine-pointed needles, photolithography using prefabricated masks, photolithography using dynamic micromirror devices, inkjet printing, or electrochemical methods on microelectrode arrays.

As used herein, the term “biomarker” refers to a substance capable of specifically labeling cells or indicating state of diseases. In the context of the invention aimed at specifically labeling cells, the combination of biomarker, Allergin-1, CD34 and CD38, can be used as a LSC-specific marker. In the context of the invention aimed at diagnosing leukemia, “biomarker” refers to a substance that indicates diagnosis, disease course monitoring and prognosis of leukemia (especially AML). “Biomarker” includes polypeptides and glycoproteins. Allergin-1 is specifically and abnormally highly expressed in AML patients, compared with normal healthy subjects.

In the invention, the terms “sample”, “specimen” or “biological sample” can be equivalently replaced, including but not limited to: a variety of sample types obtained from individuals, and can be used for diagnosis or monitoring tests. Biological fluid samples encompass blood, cerebrospinal fluid (CSF), urine, and other fluid samples of biological origin. For example, the biological sample of the invention may be blood. If necessary, the sample can be processed in advance, such as concentrated or separated.

It should be understood that the terms used herein are used for the purpose of describing specific embodiments and are not intended to be limiting. In addition, any methods, devices, and materials similar or equivalent to those described herein can be used in the practice or testing of the present invention. Preferred methods, devices, and materials are described below.

Methods, Uses and Products for Diagnosis, Disease Course Monitoring and/or Prognosis of Diseases
Methods and/or Uses

As described above, the invention provides methods for diagnosing, monitoring and evaluating leukemia such as AML by detecting or quantifying expression or activity of Allergin-1. In one aspect of the invention, the invention relates to methods for identification of LSCs by detecting expression or activity of biomarker Allergin-1 in a subject or a sample from the subject, in which Allergin-1 is specifically and abnormally highly expressed in LSCs.

In one aspect of the invention, the invention relates to methods for diagnosis, disease course monitoring and prognosis of leukemia (especially AML), or determination of risk of developing leukemia (especially AML), or determination of severity of leukemia (especially AML) by detecting expression or activity of biomarker Allergin-1 in a subject or a sample from the subject, in which Allergin-1 is specifically and abnormally highly expressed in AML patients. In some embodiments, the expression level or activity of Allergin-1 in hematopoietic stem cells of the subject is significantly higher than that in normal hematopoietic stem cells, which is an indication that the subject has leukemia or the subject is in a disease state of leukemia or the subject is at a high risk of developing leukemia or the subject has severe leukemia. In some embodiments, Allergin-1 is highly expressed in AML patients, and especially, abnormally highly expressed in the M4 or M5 subtype.

In one aspect of the invention, the invention relates to use of an agent for detecting expression level of Allergin-1 in a sample in the manufacture of a product for identifying LSCs. Preferably, the agent for detecting the expression level or activity of Allergin-1 in the sample is combined with one or more other agents. More preferably, the one or more other agents are IFN-γ.

In one aspect of the invention, the invention relates to use of an agent for detecting expression level of Allergin-1 in a sample in the manufacture of a product for diagnosis, disease course monitoring and prognosis of leukemia (especially AML). Preferably, the agent for detecting the expression level or activity of Allergin-1 in the sample is combined with one or more other agents. More preferably, the one or more other agents are IFN-γ.

In one aspect of the invention, the invention relates to use of an agent for detecting combination of Allergin-1 and at least one biomarker in the manufacture of a product for identifying LSCs. In some embodiments, the combination of Allergin-1 and at least one biomarker is Allergin-1⁺CD34⁺CD38⁻.

In one aspect of the invention, the invention relates to use of an agent for detecting combination of Allergin-1 and at least one biomarker in the manufacture of a product for diagnosis, disease course monitoring and prognosis of leukemia (especially AML). In some embodiments, the combination of Allergin-1 and at least one biomarker is Allergin-1⁺CD34⁺CD38⁻.

In embodiments of the above method or use, the term detection, determination or diagnosis includes a step of physically detecting, physically determining or physically diagnosing level or amount of a biomarker of leukemia such as AML. That is, the method includes the steps of measuring level or amount of the biomarker Allergin-1 using known methods useful for measuring the level or amount, and determining or diagnosing accordingly using the measured level or amount. In a preferred embodiment, the biomarker is determined in the form of its peptide or protein. However, the level or amount of the biomarker Allergin-1 can also be determined at nucleic acid level, for example, based on the level or amount of mRNA in a biological sample.

In embodiments of the above method or use, the progress of the disease of the subject can be detected by measuring the expression level or activity of mRNA corresponding to one or more biomarkers such as Allergin-1, or the encoded protein. The expression level of mRNA or protein of Allergin-1 can be detected in vivo, or in samples from, for example, blood or bone marrow. The expression level of mRNA and/or protein corresponding to the gene can be detected by the standard method as described above. The disease state (for example, improvement, exacerbation, or recurrence of the disease) of the subject can be monitored by comparing the level of the target protein or RNA in the subject with baseline level of target protein or RNA in the subject. For example, the expression level of Allergin-1 in the subject detected at the first time can be compared with the expression level of Allergin-1 in the subject detected at the second time later. The increase in the expression level of Allergin-1 mRNA or protein over time is an indication of progression of leukemia. The decrease in the expression level of Allergin-1 mRNA or protein over time is an indication of alleviation of leukemia.

In embodiments of the above method or use, the level of, for example, Allergin-1 protein or RNA in the subject can also be used to monitor the therapeutic effect. Generally, the baseline level of the protein or RNA of interest in the subject (for example, before the treatment) is obtained, and compared with the levels of the protein or RNA of interest measured at multiple time points after or during the treatment (for example, one day or more, several weeks or months after the treatment). The result of the comparison can indicate the effect of past treatments, and future treatments can be modified accordingly.

According to the invention, the methods disclosed herein relate to in vitro and/or in vivo methods, respectively. Preferably, the method is an in vitro method based on samples obtained from an individual and provided in vitro.

The transcript of the gene of interest can be detected using a variety of techniques known in the art. Some useful nucleic acid detection systems include preparation of a purified nucleic acid component of a sample, and direct detection of the sample, or detection after an amplification process, such as detection of Allergin-1 mRNA in a bone marrow or blood tissue sample. Amplification can be performed by, for example, polymerase chain reaction (PCR), reverse transcriptase (RT), and coupled RT-PCR. The detection of nucleic acid can be achieved by, for example, detecting the purified nucleic acid component with a probe that hybridizes to the nucleic acid of interest, and involves amplification in many cases. Northern blot, dot blot, microarray, quantitative PCR, and quantitative RT-PCR are all well-known methods for detecting nucleic acids in samples. Nucleic acid amplification can also be performed by ligase chain reaction, strand displacement amplification, autonomous sequence replication, or nucleic acid sequence-based amplification. Nucleic acid can also be detected by sequencing. Primers specific to the nucleic acid of interest (for example, Allergin-1 cDNA sequence) or primers directed to the linker sequence connected to the nucleic acid of interest can be used for sequencing. Sequencing of randomly selected mRNA or cDNA sequences can provide a characterization of relative expression level of the biomarker, which is characterized through the percentage of transcripts comprising a nucleic acid sequence corresponding to the biomarker (such as Allergin-1 cDNA or mRNA sequence) in all sequenced sequences. Alternatively, the nucleic acid can be detected in situ without extraction or purification, for example by hybridization.

In embodiments of the above method or use, the determination of the level or amount of the biomarker according to the invention is performed by known methods. For example, in the case of determining the level or amount at protein level, an immunoassay such as ELISA, RIA, radial immunodiffusion, western blot, Ouchterlony immunodiffusion, rocket immunoelectrophoresis, immunohistochemical staining, immunoprecipitation assay, complement fixation test, FACS and protein chip test, as well as immunofluorescence assay, multiple immunoassay, linearity test or dot blot is performed.

According to the method or use of the invention, in a preferred embodiment, ELISA is performed. A typical example of measuring tools used to determine the level or amount of biomarkers includes protein-specific antibodies. Specific molecular antibodies are known in the art or can be easily prepared based on methods described in the art. The measurement of protein level means measuring the amount of the protein by using a measuring tool that specifically binds to the protein, such as an antibody. The measurement method may be defined to any one of the above methods. Generally, the method includes forming an antigen-antibody complex and determining the complex by a known method.

The amount of antigen-antibody complex formed can be determined by measuring signal strength of an assay label or expression label for the biomarker protein. That is, the antibody can be affixed with detection labels known in the art. The detection labels include, but are not limited to, enzymes, fluorescent markers, ligands, luminophores, microparticles, and redox molecules. Examples of enzymes that can be used as detection labels include, but are not limited to, D-glucosidase, urease, peroxidase, alkaline phosphatase, acetylcholinesterase, glucose oxidase, hexokinase, GDP enzyme, RNase, luciferase, phosphofructokinase, phosphoenolpyruvate carboxylase, aspartate aminotransferase and phosphoenolpyruvate decarboxylase. Examples of fluorescent markers include, but are not limited to, fluorescein isothiocyanate, Rhodamine, phycoerythrin, phycocyanin, allophycocyanin, and fluorescamine. Examples of ligands include, but are not limited to, biotin, avidin, and derivatives of biotin and avidin.

In embodiments of the above method or use, for example, the target protein can be immunodetected with one or more antibodies. In immunoassays, an antibody that has specific binding affinity for the biomarker or a second antibody that binds to the antibody can be labeled directly or indirectly. The antibody is not necessary to be intact, and a variable domain of the antibody or artificial mimic thereof (such as a single-chain antibody) is sufficient. Suitable labels include, but are not limited to, radionuclides (e.g., 1251, 1311, 355, 3H, 32P, 33P or 14C), fluorescent groups (e.g., fluorescein, FITC, Peridinin-Chlorophyll-Protein Complex, Rhodamine or PE), luminescent groups, compounds or enzymes that absorb light at a certain wavelength (such as alkaline phosphatase or horseradish peroxidase). The antibody can be indirectly labeled by binding to biotin and then detecting using avidin or streptomycin labeled with the above molecule. Methods for detecting or quantifying a label based on the nature of the label are known in the art. Examples of detectors include, but are not limited to, X-ray films, radiometers, scintillometers, spectrophotometers, colorimeters, fluorometers, photometers, and densitometers. A combination of these methods known in the art (including “multi-layer” detection) can be used to increase sensitivity of the detection.

In embodiments of the above method or use, for example, the immunoassay for detecting the target protein can be performed in various known ways, including a sandwich method, a competition method (competitive RIA), or a bridge immunoassay. The method for detecting a target protein generally includes contacting the biological sample with an antibody that binds to the protein, and detecting the binding of the protein to the antibody. For example, an antibody having a specific binding affinity for Allergin-1 can be immobilized on a solid substrate by any of various methods known in the art, and then the antibody can be exposed to the biological sample.

In other embodiments, a “sandwich” detection method in which a capture antibody is immobilized on a solid substrate is used to detect the level of the protein of interest. The solid substrate can be in contact with the biological sample, such that any protein of interest in the sample can bind to the immobilized antibody. A “detection” antibody that has specific binding affinity for the protein of interest can be used to determine the level of the protein of interest bound to the above capture antibody, using the method as described above. It should be understood that in these sandwich assays, the capture antibody should not bind to the same epitope as the epitope bound by the detection antibody (or in the case of using a polyclonal antibody, the range of the epitopes bound by the capture antibody should not be the same as the range of the epitopes bound by the detection antibody). Therefore, if a monoclonal antibody is used as the capture antibody, the detection antibody can be another monoclonal antibody, and the epitope bound by the detection antibody is either physically completely separated from or only partially overlapped with the epitope bound by the monoclonal capture antibody. The detection antibody can also be a polyclonal antibody that binds to the epitope bound by a non-capturing monoclonal antibody or to the epitope other than the epitope bound by the monoclonal capture antibody. If a polyclonal antibody is used as the capture antibody, the detection antibody can be a monoclonal antibody, which binds to an epitope either physically completely separated from or partially overlapped with any epitope bound by the polyclonal capture antibody. The detection antibody can also be a polyclonal antibody that binds to the epitope bound by a non-capturing polyclonal antibody or to the epitope other than the epitope bound by the polyclonal capture antibody. Sandwich detection can be can be performed as a sandwich ELISA, a sandwich western blot or a sandwich immunomagnetic detection method.

Suitable solid substrates to which antibodies (such as capture antibodies) can be bound include, but are not limited to, microplates, tubes, membranes such as nylon membranes or nitrocellulose membranes, and beads or particles (such as agarose, cellulose, glass, polystyrene, polyacrylamide, magnetic or magnetizable beads or particles). When using an automatic immune detection system, magnetic particles or magnetizable particles are particularly suitable.

Other techniques for detecting the peptide of interest include mass spectrophotometric techniques such as electrospray ionization (ESI) and matrix-assisted laser desorption ionization (MALDI).

In embodiments of the above method or use, preferably, the agents described above include but are not limited to agents suitable for detecting the expression level of Allergin-1 by RT-PCR, real-time quantitative PCR, immunoassay (such as ELISA, RIA, multiple immunoassay, immunofluorescence assay, western blot, or dot blot), in situ hybridization or chip technology. More preferably, the agents include primers/probes for Allergin-1 gene, molecular beacons, RNAi, or antibodies and/or ligands against Allergin-1 protein, zinc finger, and small molecule compounds.

In embodiments of the above method or use, preferably, the products described above include but are not limited to chips, agents, test papers, formulations, kits or high-throughput screening platforms.

In embodiments of the above method or use, preferably, in one embodiment, the method or use according to the invention includes following steps:
a) collecting a biological sample of a subject, preferably bone marrow cells;
b) detecting expression level of Allergin-1 in CD34⁺CD38⁻ cells in the above biological sample; and
c) comparing the level or amount of the biomarker Allergin-1 detected in the biological sample with a reference level or amount of the biomarker.

Preferably, the reference label is: 1) an average label obtained from a population not suffering from leukemia such as AML; and/or 2) an average or median level from a group of individuals including patients having leukemia such as AML.

It should be understood that the expression of transcript of any gene of interest or protein of interest according to the invention can be easily detected by using one or more of the above techniques.

### Products for Diagnosis or Disease Course Monitoring

In one aspect of the invention, the invention provides a product for diagnosis, disease course monitoring and prognosis of leukemia (especially AML). The product includes a chip, a formulation or a kit for detecting expression level of Allergin-1.

Preferably, the invention provides a detection kit for diagnosis, disease course monitoring and prognosis of leukemia (especially AML). Further, the detection kit includes at least one pair of primers for specifically amplifying Allergin-1 gene. Preferably, the primer has the sequence of SEQ ID NO: 18, 19, 20, 21, 22 or 23 shown in Table 3.

TABLE 3
Allergin-1 Q-PCR primers (5′→3′)

| | | |
|---|---|---|
| SEQ ID | Forward | CCCAAGTTACCAGCTGTTCAAA |
| NO. 18 | | |
| SEQ ID | Reverse | TATATGTCGGTCTGTTTCTGTTTGAAT |
| NO. 19 | | |
| SEQ ID | Forward | TAGGATTATCACCACCAGCAACAG |
| NO. 20 | | |
| SEQ ID | Reverse | AGCTGTATTGGATTGTGAGGCA |
| NO. 21 | | |
| SEQ ID | Forward | AAATGCAAAGCCCAAGTTACCA |
| NO. 22 | | |
| SEQ ID | Reverse | AATTGATGGGCAGCGAGC |
| NO. 23 | | |

Preferably, the detection kit of the invention for detecting a biomarker comprises an antibody specific to the above protein, and also a second antibody coupled to a label for developing with a substrate, such as an enzyme; a chromogenic substrate solution that induces development with the label; a washing solution; and a stop solution for the enzymatic reaction. It may also comprise a suitable microplate, a standard solution and a protocol. The (first) antibody specific for the biomarker described above and below can be coupled to a label by itself instead of the second antibody coupled to the label.

The detection kit of the invention for detecting a biomarker can be used to diagnose leukemia by qualitatively or quantitatively analyzing an antigen through an antigen-antibody binding reaction, which can be detected by a conventional method such as ELISA (enzyme-linked immunosorbent assay) or a sandwich assay. For example, the detection kit for detecting the detection biomarkers as described above or below can be set in such way that ELISA for reacting with a recombinant monoclonal antibody protein is performed by using a 96-well microtiter plate coated with an analyte and a control on its surface. As a receptor for the antigen-antibody binding reaction, polyethylene resin or polystyrene resin, nitrocellulose membrane, or glass slide can be used. Conventional labels for development such as HRP (horseradish peroxidase), alkaline phosphatase, colloidal gold, fluorescent labels such as fluorescein, FITC (fluorescein poly-L-lysine-isothiocyanate) or RITC (Rhodamine-B isocyanate) or dyes can be preferably used as a label for the second antibody conjugate or the first antibody conjugate. Other methods for determining the amount of biomarkers are also considered, especially RIA (radial immunoassay), western blot on polyacrylamide gel, immunoblot and immunohistochemical staining. As well known in the art, the detection kit is adjusted for the specific method considered for the quantitative determination of the biomarker.

As indicated above, in the case of determining the level or amount of the biomarker at protein level, the agent is, for example, an antibody. Optionally, in the case of determining the level or amount at nucleic acid level, the agent can be a nucleic acid molecule.

Preferably, in embodiments of the above product, the detection kit of the invention can be used to detect the expression of Allergin-1. Preferably, the kit comprises a detection effective amount of an agent for detecting Allergin-1 gene and one or more substances selected from the group consisting of containers, instructions for use, positive controls, negative controls, buffers, adjuvants or solvents, for example, a solution for suspending or fixing cells, a detectable label or tag, a solution for facilitating hybridization of nucleic acid, a solution for lysing cells, or a solution for nucleic acid purification.

More preferably, the kit of the invention may also be accompanied by an instruction for use of the kit, which describes how to use the kit for detection, and how to use the detection results to judge the development of leukemia and select the therapeutic regimen.

In embodiments of the above product, using the kit of the invention, Allergin-1 can be detected by various methods (including but not limited to) selected from the group consisting of real-time quantitative reverse transcription PCR, biochip detection method, southern blot, or northern blot or in situ hybridization. The detection method can be adjusted and modified by a person of ordinary skill in the art according to actual conditions and needs.

In one aspect of the invention, the array of the invention for detecting biomarkers may be a microtiter plate.

In addition, the invention relates to a computer-implemented method for diagnosis of leukemia or determination of risk of developing leukemia, comprising following steps:
a) obtaining a biomarker for leukemia as defined herein, that is, data of level or amount of Allergin-1 measured in a biological sample of a subject and optionally a control;
b) calculating the data in step a) by comparing the level or amount of the biomarker in the sample with a limit value obtained from a database or a limit value obtained from a measured level or a measured amount as a limit control; and
c) analyzing and identifying biomarkers whose level or amount has increased above or decreased below the limit level.

Optionally, the method further includes a step of displaying the result of each biomarker or only the result of a positive or negative diagnosis or risk assessment in the form of, for example, colored and highlighted display in an output unit. For example, the analysis is performed by principal component analysis. The analysis may be accompanied by error analysis.

The invention also relates to a computer medium or computer program product having computer-executable instructions for performing the steps of the method according to the invention.

In some embodiments, the comparison of the measured value and the reference value includes calculating fold change between the measured value and the reference value, thereby correspondingly identifying whether the measured value is above or below the limit value.

Methods and/or Uses for Treating and/or Alleviating Diseases

The invention provides a method or use for treating and/or alleviating leukemia (for example, AML) in a subject. Specifically, the treatment and/or alleviation of leukemia (for example, AML) is (are) achieved by affecting proliferation, differentiation, and in vivo engraftment (or in vivo growth) of LSCs and AML cells by regulating the expression of Allergin-1 in these cells. In one aspect of the invention, the invention relates to use of an agent in the manufacture of a product for treatment of leukemia (especially AML) in a subject. Preferably, the agent is a modulator (such as inhibitor/antagonist) for expression or activity of Allergin-1. Preferably, the product is a medicament or a combination of medicaments.

In embodiments in the method or use described above, the regulation of the expression or activity of Allergin-1 can be achieved by modulators (such as inhibitors/antagonists) for expression or activity of Allergin-1, such as substances that reduces the expression of nucleic acid encoding Allergin-1, reduces the level of Allergin-1 protein, or inhibits the activity of Allergin-1, for example, substances that reduce the activity of Allergin-1 protein, reduce the stability of Allergin-1 gene or protein, down-regulate the expression of Allergin-1, reduce the effective acting time of Allergin-1 protein, or inhibit the transcription and translation of Allergin-1.

Preferably, the inhibitors and/or antagonists described above include, but are not limited to, nucleic acid inhibitors, protein inhibitors, proteolytic enzymes, protein binding molecules; and combination thereof. The nucleic acid inhibitor is selected from interfering molecules that target Allergin-1 or its transcript and can inhibit expression or transcription of Allergin-1 gene, including shRNA (small hairpin RNA), small interfering RNA (siRNA), dsRNA, microRNA, or constructs capable of expressing or forming the shRNA, siRNA, dsRNA, microRNA. The protein binding molecule is selected from substances that specifically bind to Allergin-1 protein, such as antibodies or ligands capable of inhibiting the activity of Allergin-1 protein; competitors for the Allergin-1 ligand binding site, including Allergin-1 receptors and ligand binding fragments thereof, soluble truncated Allergin-1 receptors, soluble Allergin-1 receptor fusion proteins, such as Allergin-1 fusion proteins or ligand fusion proteins containing Fc portion of IgG immunoglobulins; peptidomimetics; peptide inhibitors; small molecule compounds; and combination thereof.

More preferably, the invention also relates to the use of RNA interference (RNAi) to inhibit the expression of Allergin-1. Any type of RNA interference sequence can be used in the invention. Non-limiting examples thereof include short interfering RNA (siRNA) molecules or short hairpin RNA (shRNA). A variety of algorithms can be used for RNAi sequence design. More preferably, the modulator (such as inhibitor/antagonist) is shRNA; especially preferably, the shRNA has the sequence shown in SEQ ID NO: 12, 13, 14, 15, 16 or 17 in Table 2.

TABLE 2
shRNA sequences

| | Sense Oligo (5′→3′) |
|---|---|
| Target sense sequence | Antisense Oligo (5′→3′) |
| GCCCAAGTTACCAGCTGTT | TGCCCAAGTTACCAGCTGTTTTCAAGAGAAACAGCTGGT |
| (SEQ ID NO: 24) | AACTTGGGCCTTTTTTC (SEQ ID NO: 12) |
| | TCGAGAAAAAAGGCCCAAGTTACCAGCTGTTTCTCTTGA |
| | AAACAGCTGGTAACTTGGGCA (SEQ ID NO: 13) |
| GCTACTACTTCCAGGGTTA | TGCTACTACTTCCAGGGTTATTCAAGAGATAACCCTGGA |
| (SEQ ID NO: 25) | AGTAGTAGCTTTTTTC (SEQ ID NO: 14) |
| | TCGAGAAAAAAGCTACTACTTCCAGGGTTATCTCTTGAA |
| | TAACCCTGGAAGTAGTAGCA (SEQ ID NO: 15) |
| GCTGGTGGTGATAATCCTA | TGCTGGTGGTGATAATCCTATTCAAGAGATAGGATTATC |
| (SEQ ID NO: 26) | ACCACCAGCTTTTTTC (SEQ ID NO: 16) |
| | TCGAGAAAAAAGCTGGTGGTGATAATCCTATCTCTTGAAT |
| | AGGATTATCACCACCAGCA (SEQ ID NO: 17) |

In addition, the sequence of interest can be selected as a sequence having low homology with other variants or gene sequences. The effect of the RNAi molecule can be evaluated by introducing or expressing the RNAi sequence in cells expressing the product of the gene of interest. The substantial change in mRNA or protein level of the product of the gene of interest is an indication of the effectiveness of the RNAi molecule in inhibiting the expression of the gene. Methods for expressing RNAi molecules in cells are well known in the art, and include, for example, lentiviral vectors.

More preferably, the modulator (such as inhibitor/antagonist) is a ligand binding competitor. The signal of Allergin-1 can also be suppressed by administering a competitor that binds to the Allergin-1 ligand. This can be achieved, for example, by administering a soluble fragment of the extracellular domain of Allergin-1, which is optionally coupled to a carrier protein (such as IgG immunoglobulin known in the art). For example, administration of Allergin-1-Fc fusion protein formed by an Allergin-1 fragment and a Fc portion of human IgG1 has been disclosed. The Fc portion of IgG in the fusion protein can be from any IgG subclass (such as IgG1, IgG2, IgG3 and IgG4). It is important that the treatment of human does not necessarily require the administration of a wild-type human Allergin-1 fragment. Other Allergin-1 fragments from other mammals can also be used, and one or more amino acid substitutions can be introduced therein, as long as the fragments retain the ability to compete with endogenous human Allergin-1 for binding to the ligand.

In embodiments of the above method or use, other means for treating leukemia such as AML include administering binding agents, such as proteins, peptides and/or antibodies or portions thereof (such as, Fab, F(ab′)2, Fv or single chain Fv fragment), which interact with the therapeutic target, for example bind to and/or neutralize the therapeutic target. The administration of an anti-Allergin-1 binding agent, such as an anti-Allergin-1 antibody, to patients with leukemia, such as AML, can alleviate the symptoms of the disease by inhibiting and/or antagonizing Allergin-1. The antibody can be an isolated antibody. In one embodiment, the antibody is an antagonistic antibody. In another embodiment, the antibody is a neutralizing antibody. In a further embodiment, the antibody modulates, reduces, and/or inhibits one or more Allergin-1 related activities, including but not limited to modulates, reduces and/or inhibits the interaction of Allergin-1 with Allergin-1 ligand; modulates, reduces and/or inhibits Allergin-1 mediated signal transduction; and modulates, reduces and/or inhibits the expression of genes activated by Allergin-1. The anti-Allergin-1 antibody of the invention may include, for example, an antibody that specifically binds to Allergin-1 and/or an antibody that binds to a membrane-bound form of Allergin-1 receptor without activating the Allergin-1 receptor. The anti-Allergin-1 antibody of the invention may also include single domain antibodies from any species. Alternative binding domain polypeptides can also be used to inhibit and/or antagonize the activity or signal transduction of Allergin-1.

More preferably, the modulator (such as inhibitor/antagonist) is an antibody that specifically binds to Allergin-1; preferably, for example, the specific antibody includes monoclonal antibodies, polyclonal antibodies, neutralizing antibodies, antigen-binding fragments, conjugated Allergin-1 antibodies.

In embodiments of the above method or use, preferably, the treatment is immunotherapy.

In embodiments of the above method or use, preferably, the treatment is gene therapy. For example, the modulator (e.g. inhibitor/antagonist) for Allergin-1 can be directly administered to the subject by methods such as injection. Alternatively, the expression unit (such as an expression vector or virus, or siRNA or shRNA) carrying the modulator (e.g. inhibitor/antagonist) for Allergin-1 can be delivered to the target through a certain route, and expresses the active modulator (such as inhibitor/antagonist) for Allergin-1, depending on the type of the inhibitor, which are well known to those skilled in the art.

In embodiments of the above use, the method for the treatment of leukemia further includes administering to the subject a therapeutically effective amount of the modulator (such as inhibitor/antagonist) for expression or activity of Allergin-1 in combination with one or more therapeutic agents.

Preferably, the one or more therapeutic agents are IFN-γ.

In one aspect of the invention, the present invention relates to use of the modulator in the manufacture of a medicament, a formulation or a therapeutic kit, wherein the medicament, the formulation or the therapeutic kit is used in the treatment and/or alleviation of leukemia (for example, AML) by affecting proliferation, differentiation, and in vivo engraftment (or in vivo growth) of LSCs and AML cell by regulating the expression of Allergin-1 in these cells.

Preferably, in embodiments of the above use, the medicament comprises the modulator (such as inhibitors/antagonists) for expression or activity of Allergin-1 described above or pharmaceutically acceptable derivatives or functional analogues thereof and pharmaceutically acceptable adjuvants, excipients, carriers, solvents or combination thereof.

It should also be recognized that the modulator (such as inhibitors/antagonists) or functional analogues disclosed in the invention may exist in free form for treatment, or may exist in the form of pharmaceutically acceptable derivatives thereof if appropriate.

Some non-limiting embodiments of pharmaceutically acceptable derivatives include pharmaceutically acceptable prodrugs, salts, esters, and salts of these esters, or any additional adducts or derivatives that can directly or indirectly provide the aforementioned agonists, promoters, antagonists or inhibitors or functional analogues or metabolites or residues thereof of the invention when administered to patients in need thereof.

An effective amount of the modulator (such as inhibitor/antagonist) described above can be administered to prevent or treat the disease. The appropriate dosage of the modulator (such as inhibitor/antagonist) described above, such as anti-Allergin-1 antibodies or antigen-binding fragments thereof, can be determined according to the type of disease to be treated, the type of the modulator (such as inhibitor/antagonist), the severity and course of the disease, the severity of the treatment, the clinical symptoms of the individual, the clinical history of the individual and the response to the treatment. In some embodiments, the combination therapy of the modulator (such as inhibitor/antagonist) described above and other drugs is synergistic and therefore the effective dose in the combination is reduced, relative to the effective dose of the modulator (such as inhibitor/antagonist) alone.

Generally, the therapeutically effective amount of the modulator (such as inhibitor/antagonist) such as Allergin-1 antibodies or antigen-binding fragments thereof administered to human herein is about 0.0001 mg/kg to about 100 mg/kg (e.g., about 0.01 mg/kg, about 0.1 mg/kg, about 0.5 mg/kg, about 1 mg/kg) of body weight of the patient, regardless of one or more administrations. In some embodiments, the antibody or antigen-binding fragment thereof is used at about 0.01 mg/kg, about 0.015 mg/kg, about 0.1 mg/kg, about 0.15 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 5 mg/kg, about 10 mg/kg, about 15 mg/kg, about 20 mg/kg, 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg or about 100 mg/kg. In some embodiments, the antibody or antigen-binding fragment thereof is administered at a dose of about 50 mg/kg or less, 10 mg/kg or less, 5 mg/kg or less, 1 mg/kg or less, 0.5 mg/kg or less, or less than or equal to 0.1 mg/kg. In some embodiments, the antibody or antigen-binding fragment thereof is used at about 0.01 mg/kg to 0.2 mg/kg.

The modulator (such as inhibitor/antagonist) or functional analogues disclosed in the invention can be prepared and packaged into a bulk form, in which a safe and effective amount can be extracted, and then administered to patients in the form of powder or syrup. Alternatively, the modulator (such as inhibitor/antagonist) disclosed in the invention can be prepared and packaged into a unit dosage form, in which each physically discrete unit contains a safe and effective amount. When prepared in a unit dosage form, the modulator (such as inhibitor/antagonist) disclosed in the invention, for example, Allergin-1 antibody or antigen-binding fragment thereof is administered in a unit dose of about 1 mg to about 100 mg, preferably in a unit dose of about 5 mg, about 10 mg, 15 mg, 20 mg, 25 mg, 30 mg, 35 mg, 40 mg, 45 mg, 50 mg.

In some embodiments, the administered dose can be changed during the treatment. For example, in some embodiments, the initial administered dose may be higher than the subsequent administered dose. In some embodiments, the administered dose may vary during the treatment, depending on the response of the subject.

The modulator (such as inhibitor/antagonist) or one or more other drugs/therapeutic agents can be administered at a specific dose at multiple intervals, for example, once a day, twice or more times a day; once a week, twice or three or more times a week; once, twice or more times a month; once a week, once every two weeks, once every three weeks, once a month or once every two months, or less. In some embodiments, the administered dose can vary over the treatment. For example, in certain embodiments, the initial administered dose may be higher than the subsequent dose. In certain embodiments, the administered dose is adjusted during the treatment depending on the response of the subject. The dosage regimen can be adjusted to achieve an optimal response (e.g., a therapeutic response). For example, a single dose or multiple separate doses can be administered over a period of time.

The “pharmaceutically acceptable adjuvants, excipients, carriers, and solvents” disclosed in the invention refer to pharmaceutically acceptable materials, mixtures or solvents that are compatible with the administered dosage form or the pharmaceutical composition. Each adjuvant, when mixed, must be compatible with the other ingredients of the pharmaceutical composition, so as to avoid the interaction that would greatly reduce the efficacy of the modulator (such as inhibitor/antagonist) disclosed in the invention when administered to patients and the interaction that would result in a pharmaceutical composition that is not pharmaceutically acceptable. In addition, each adjuvant must be pharmaceutically acceptable, for example, has sufficiently high purity.

Suitable “pharmaceutically acceptable adjuvants, excipients, carriers, and solvents” will vary depending on the specific dosage form selected. In addition, the pharmaceutically acceptable adjuvants can be selected according to their specific functions in the composition. For example, certain pharmaceutically acceptable adjuvants that facilitate producing a uniform dosage form can be selected. Certain pharmaceutically acceptable adjuvants that facilitate producing a stable dosage form can be selected. Certain pharmaceutically acceptable adjuvants that facilitate carrying or transporting the compound disclosed in the invention from one organ or part of body to another organ or part of body when administered to a patient can be selected. Certain pharmaceutically acceptable adjuvants that enhance patient compliance can be selected.

The “pharmaceutically acceptable adjuvants, excipients, carriers, and solvents” used in the disclosure may include, for example, pharmaceutically acceptable liquid, gel or solid carriers, aqueous media, non-aqueous media, antimicrobial materials, penetrating materials, buffers, antioxidants, anesthetics, suspending/dispersing agents, chelating agents, diluents, adjuvants or non-toxic auxiliary substances, other components known in the art or various combinations of the above.

Suitable components may include, for example, antioxidants, fillers, binders, disintegrants, buffers, preservatives, lubricants, flavoring agents, thickeners, colorants, emulsifiers or stabilizers, such as sugar and cyclodextrin. Suitable antioxidants can include, for example, methionine, ascorbic acid, EDTA, sodium thiosulfate, platinum, catalase, citric acid, cysteine, mercaptoglycerol, mercaptoglycolic acid, sorbitan, butyl fennel ether, butylated hydroxytoluene and/or propyl gallate.

In addition, pharmaceutically acceptable excipients or carriers may include, for example, aqueous vehicles, such as sodium chloride injection, Ringer's injection, isotonic dextrose injection, sterile water injection, or dextrose and lactate Ringer's Injections, non-aqueous vehicles such as fixed oils, plant sources, cottonseed oil, corn oil, sesame oil or peanut oil, antibacterial agents with bacteriostasis or bacteriostatic concentration, isotonic agents (such as sodium chloride or glucose), buffers (such as phosphate or citrate buffers), antioxidants (such as sodium bisulfate), local anesthetics such as procaine hydrochloride, suspending agents and dispersing agents, such as sodium carboxymethyl cellulose, hydroxypropyl methyl cellulose or polyvinylpyrrolidone, emulsifiers, such as polysorbate 80 (TWEEN-80), chelating agent such as EDTA (ethylenediamine tetraacetic acid) or EGTA (ethylene glycol tetraacetic acid), ethanol, polyethylene glycol, propylene glycol, sodium hydroxide, hydrochloric acid, citric acid or lactic acid. The antimicrobial agent that can be used as a carrier can be added to the pharmaceutical composition in a multi-dose container that includes phenol or cresol, mercury, benzyl alcohol, chlorobutanol, methylparaben and propylparaben, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate or cyclodextrin.

Preferably, suitable pharmaceutically acceptable adjuvants include following types of adjuvants: diluents, fillers, binders, disintegrants, lubricants, glidants, granulating agents, coating agents, wetting agents, solvents, co-solvents, suspending agents, emulsifiers, sweeteners, flavoring agents, taste masking agents, coloring agents, anti-caking agents, humectants, chelating agents, plasticizers, tackifiers, antioxidants, preservatives, stabilizers, surfactants and buffers. The skilled person can recognize that certain pharmaceutically acceptable adjuvants can provide more than one function and provide alternative functions, depending on the amount of the adjuvants present in the formulation and the type of other adjuvants present in the formulation.

The modulators (such as inhibitors/antagonists) or functional analogues thereof disclosed in the invention are generally formulated into a dosage form suitable for administration to a patient through a desired route. For example, the dosage form includes those suitable for the following routes of administration: (1) oral administration, such as tablets, capsules, caplets, pills, lozenges, powders, syrups, elixirs, suspensions, solutions, emulsions, granules and cachets; (2) parenteral administration, such as sterile solutions, suspensions and freeze-dried powders; (3) transdermal administration, such as transdermal patches; (4) rectal administration, such as suppositories; (5) inhalation, such as aerosols, solutions and dry powders; and (6) topical administration, such as creams, ointments, lotions, solutions, pastes, sprays, foams and gels.

In some embodiments, the modulators (such as inhibitors/antagonists) or functional analogues thereof, such as Allergin-1 antibodies or antigen-binding fragments thereof, are administered through implantation, inhalation, intrathecal, intraventricular or intranasal, intravenous, intramuscular, subcutaneous, topical, oral, transdermal, intraperitoneal, intraorbital, or oral administration, preferably intravenous administration.

In some embodiments, the above methods or uses may further include an additional therapy. The additional therapy may be radiation therapy, surgery, chemotherapy, gene therapy, DNA therapy, viral therapy, RNA therapy, immunotherapy, bone marrow transplantation, nanotherapy, monoclonal antibody therapy, or a combination thereof. The additional therapy can employ the form of adjuvant or neoadjuvant therapy. In some embodiments, the additional therapy is administration of small-molecule enzyme inhibitors or anti-metastatic agents. In some embodiments, the additional therapy is administration of side-effect limiting agents (e.g., agents intended to reduce the occurrence and/or severity of side effects of the treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma radiation.

The treatment method or use of the invention can be used to remove tumors before or after surgery, and can be used before, during or after radiotherapy. In some embodiments, the treatment method or use of the invention is applied to the patient who has not previously been treated with a biotherapeutic agent or chemotherapeutic agent. In other embodiments, the treatment method or use of the invention is applied to the patient who has failed to achieve a sustained response after previous treatment with a biotherapeutic agent or chemotherapeutic agent (i.e., has been subjected to treatment).

Medicaments, Combination of Medicaments and/or Methods for Screening Medicaments

In one aspect of the invention, the invention provides a medicament, a composition or a therapeutic kit for treating leukemia, wherein the medicament, the composition or the therapeutic kit comprises a modulator (such as inhibitor/antagonist) for functional expression of Allergin-1, and/or other type of drug compatible with the modulator (such as inhibitor/antagonist) and pharmaceutically acceptable adjuvants, excipients, carriers, solvents, or combination thereof.

Preferably, in embodiments of the above medicament or composition, the modulator (such as inhibitor/antagonist) is as defined in the section “Methods and/or uses for treating and/or alleviating diseases” above.

Preferably, in embodiments of the above medicament or composition, the pharmaceutically acceptable adjuvants, excipients, carriers, solvents or combination thereof are as defined in the section “Methods and/or uses for treating and/or alleviating diseases” above.

Preferably, the other type of drug is IFN-γ.

Preferably, the medicament or composition of the invention can also be used in combination with other drugs for the treatment of leukemia. The other therapeutic drugs can be administered simultaneously with the main active ingredient, or even administered simultaneously in the same composition. The other therapeutic drugs can also be administered alone in a separate composition or in a dosage form different from the main active ingredient.

In one aspect of the invention, the invention provides a method for screening candidate drugs for treatment of leukemia (especially AML) by biomarker Allergin-1. Preferably, the method includes treating a system expressing or containing Allergin-1 gene with a substance to be screened; and detecting expression level of the Allergin-1 gene in the system; wherein, if the substance to be screened can reduce the expression level of the Allergin-1 gene, the substance to be screened is identified to be a candidate drug for treatment of leukemia (especially AML). More preferably, the system described above includes (but is not limited to): cell system, subcellular system, solution system, tissue system, organ system or animal system. The candidate drug includes (but is not limited to): interfering molecules designed for Allergin-1 gene or upstream or downstream genes thereof, nucleic acid inhibitors, small molecule compounds.

### Mode of Carrying Out the Invention

Those skilled in the art can understand other objects of the present invention through the description in the context and the examples. The examples described below are exemplary, and are only used to illustrate the present invention, but should not be construed as limiting the present invention. Where specific techniques or conditions are not indicated in the examples, the procedures shall be carried out in accordance with the techniques or conditions described in the literature in the art or in accordance with the product specification. The agents or instruments used without indicating the manufacturer are all conventional products that are commercially available. In all quantitative experiments in the following examples, three repeated experiments were set up, and the results were averaged.

In the following examples, anti-human Allergin-1 antibody (clone #767727, mouse IgG1) was purchased from R&D Systems. Phage display technology was used to generate human Allergin-1 antibody (human IgG1) targeting the human Allergin-1L (Allergin-1 long form) (GenBank: human ALLERGIN-1L, AB542950).

### Example 1

### TARGET (Therapeutically Available Research to Generate Effective Treatments) Database Analysis

### Experimental Method:

The correlation between the expression level of Allergin-1 and the prognosis of AML and the expression of Allergin-1 in each subtype of AML patients were analyzed based on the RNA Sequencing data (FPKM) of AML patients in the TARGET database (https://ocg.cancer.gov/programs/target/, accessed Dec. 9, 2019). According to the median of expression value of Allergin-1, 87 AML patients with M4/M5 subtype were divided into a high expression group and a low expression group, and Kaplan-Meier survival analysis of different groups of AML patients was performed.

### Experimental Results:

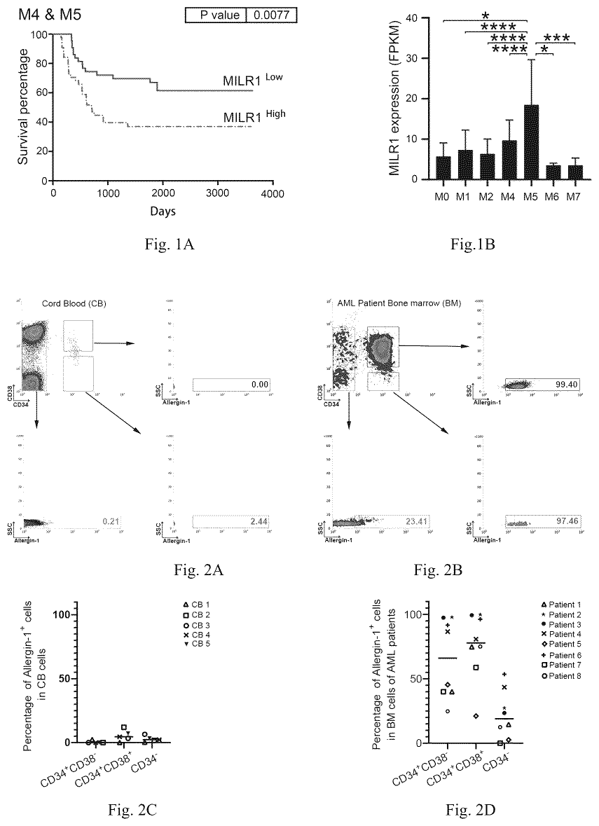

By analysis of the expression of cell surface biomarkers in AML patients through the TARGET AML database, it was found that there is a significant correlation between the expression of Allergin-1 in AML patients with M4/M5 subtype and the survival rate of AML patients; the expression of Allergin-1 in patients with poor prognosis is significantly increased (FIG. 1A); and the expression of Allergin-1 in AML patients with M5 subtype is higher than that in patients with other subtypes (FIG. 1B). Among them, in FIG. 1B, M0-M5, *p=0.028; M1-M5, ****p<0.0001; M2-M5, ****p<0.0001; M4-M5, ****p<0.0001; M5-M6, *p=0.0274; M5-M7, ***p=0.0005. Error bars, s.e.m.

The results indicate that Allergin-1 can be used for disease course monitoring and prognosis of AML.

### Example 2

### Detection of the Expression of Allergin-1 in HSCs, LSCs, Primary AML Cells and AML Cell Lines of Patients, and Detection of Self-Renewal of LSCs

### Experimental Method:

Bone marrow samples of patients with primary AML (see Table 1 for details) and cord blood of full-term healthy newborns were provided by relevant hospitals. All samples were included in the experiment after the permission of the ethics committee and the informed consent of volunteers after being informed of relevant matters.

TABLE 1
Sample of 8 primary patients used in the invention

| AML | | | FAB | Primary/ | | | | CD34⁺ | Allergin-1⁺ |
|---|---|---|---|---|---|---|---|---|---|
| patients | Age | Gender | sutype | Secondary | Specimen | Karyotype | Molecular alterations | (%) | (%) |
| 1 | 65 | Male | M2 | primary | BM | Normal | | 16.65 | 11.34 |
| 2 | 62 | Male | M5 | primary | BM | 46, XY, t: (9, 11) (p22; q23) | MLL/AT9, DNMT3A | 12.5 | 10.3 |
| 3 | 68 | Male | M5 | primary | BM | Normal | IDH1, DNMT3A, CEBPA | 85.98 | 48.9 |
| 4 | 71 | Male | M4 | primary | BM | 46, XY, der(17)(p11)[1]/46, | DNMT3A, RUNX1, | 20.28 | 75.73 |
| | | | | | | XY[19] | SF3B1, FLT3-ITD | | |
| 5 | 87 | Male | M2 | primary | BM | Normal | | 63.65 | 13.68 |
| 6 | 46 | Female | M5 | primary | BM | 46, XX, t (9, 11) (p22; q23) | MLL/AT8 | 3.79 | 47.03 |
| 7 | 67 | Male | M2 | Primary | BM | Sample sent out inspection | | 48.28 | 4.18 |
| 8 | 64 | Female | M2 | primary | BM | 46, XX, t (6, 11)(q27, q23) | NRAS | 94.06 | 14.08 |

HL 60 Thp1 MV4-11 and U937 were all purchased from ATCC. Mononuclear cells from cord blood and bone marrow of AML patients were separated by using Ficoll-Paque cell density gradient separation solution (Pharmacia Biotech, Uppsala, Sweden). The expression levels of Allergin-1 in CD34⁺CD38⁻ cord blood enriched with normal HSCs or LSCs and bone marrow cells of patients with primary AML were detected by flow cytometry. The human AML cell lines were routinely cultured in DMEM complete medium (containing 10% FBS, 100 U/ml penicillin and 100 U/ml streptomycin), and placed in an incubator at 37° C., 5% CO₂ and saturated humidity, and passaged once every 3-4 days. The expression levels of Allergin-1 in the above AML cell lines were detected by flow cytometry. 3000 CD34⁺CD38⁻ Allergin-1⁺ and CD34⁺CD38⁻ Allergin-1⁻ cells were sorted from the bone marrow cells of patients with primary AML, mixed thoroughly with artificial hematopoietic colony methylcellulose medium (M3534, Stem Cell Technologies, Vancouver, BC, Canada), then added to a 35 mm cell culture dish and cultured in an incubator at 37° C., 5% CO₂ and saturated humidity. After 6 days, colony forming units with >50 cells were counted by an inverted microscope. The self-renewal ability of the above cells was detected. The antibodies used are Human Allergin-1 antibody, PE or FITC-conjugated anti-human CD34 (Cone #8G12, Becton Dickinson Company, BD Biosciences) and PE-Cyanine7-conjugated anti-human CD38 (Clone #HIT2, eBioscience), respectively.

### Experimental Results:

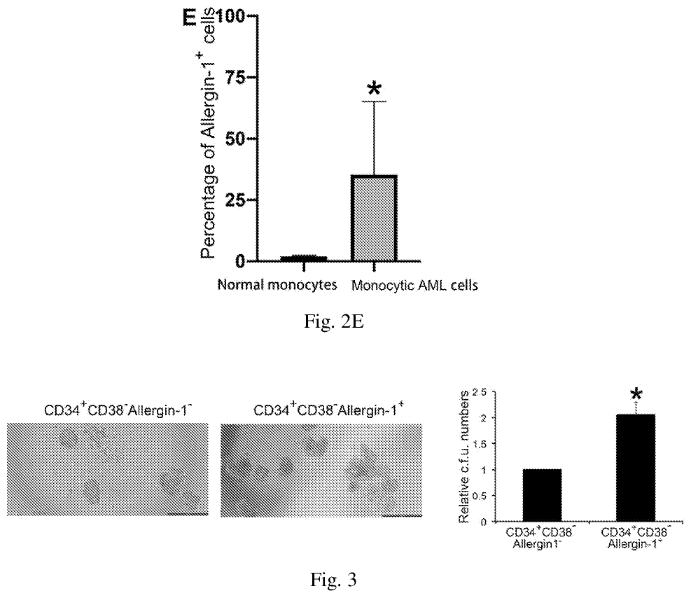

CD34⁺CD38⁻ cells are enriched with HSCs or LSCs. Therefore, the present invention collects healthy human cord blood and bone marrow cells of patients with primary AML (Table 1) to detect the expression level of Allergin-1 in the above CD34⁺CD38⁻ cells. It was found that Allergin-1 is not expressed in CD34⁺CD38⁻ cells of healthy human cord blood (FIGS. 2A and 2B), but is abnormally and significantly highly expressed in CD34⁺CD38⁻ cells of bone marrow of patients with primary AML (FIGS. 2C and 2D), suggesting Allergin-1 can be used to identify normal HSCs and LSCs.

It can be known from FIGS. 2C and 2D that the expression level or activity of Allergin-1 in hematopoietic stem cells is about 3 times to about 100 times of that in normal hematopoietic stem cells, with an average of about 57 times.

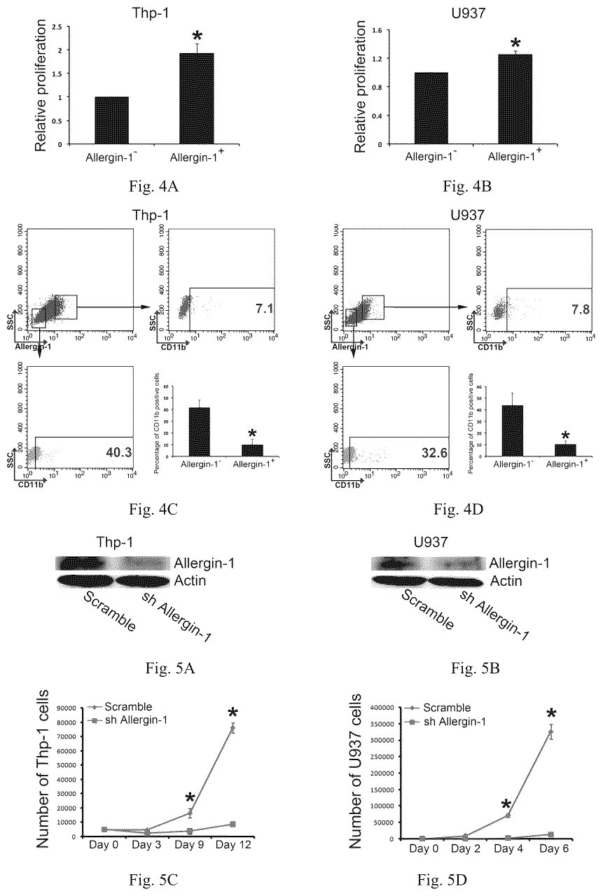

CD34⁺CD38⁻Allergin-1⁺ and CD34⁺CD38⁻Allergin-1⁻ cells were sorted from the bone marrow cells of patients with primary AML for CFU formation experiments. It was found that the CFU forming ability of the above CD34⁺CD38⁻Allergin-1⁺ cells is significantly higher than that of CD34⁺CD38⁻Allergin-1⁻ cells (FIG. 3, (n=3), *p<0.05.), suggesting that the self-renewal ability of CD34⁺CD38⁻Allergin-1⁺ cells is significantly higher than that of CD34⁺CD38⁻ Allergin-1⁻ cells.

### Example 3

### Detection of the Effect of Allergin-1 on the Proliferation and Differentiation of AML Cells

### Experimental Method:

Construction of plasmid with Allergin-1 knockdown and transfection of AML cells: 3 pairs of specific interference sequences targeting Allergin-1 gene (see Table 14 for details) and 1 pair of non-specific interference sequences were synthesized, and transfected Thp-1 and U937 cells. Virus particles were labeled with green fluorescent protein (GFP). After 72 hours of transfection, GFP⁺ cells were sorted by flow cytometry, and the efficiency of Allergin-1 knockdown in Thp-1 and U937 cells was verified by western blot assay with anti-Allergin-1 antibody (ab177744, abeam).

Drawing of cell growth curve and detection of cell differentiation: 72 hours after transfection of shRNA, GFP-positive Thp-1 and U937 cells were sorted from the above cells and placed in a 96-well U-shaped plate (Corning Incorporated Costar 3799, Corning), with 0.2 ml containing 3000 cells per well, and continuously cultured for 6 or 12 days. Cells were collected and counted every 48 or 72 hours and analyzed for the effect of Allergin-1 on the proliferation of Thp-1 and U937 cells. The expression level of CD11b (Clone #M1/70, BioLegend) in Thp-1 (12 days) and U937 cells (6 days in culture) was detected by flow cytometry to explore the effect of Allergin-1 on the differentiation of Thp-1 and U937 cells.

### Experimental Results:

The indicators for proliferation and differentiation of Thp-1 and U937 cells of Allergin-1⁺ or Allergin-1⁻ AML cell lines were detected. It was found that the proliferation ability of Allergin-1⁺AML cells is significantly higher than that of control Allergin-1⁻ cells, (n=3), *p <0.05 (FIGS. 4A and 4B), and the expression level of CD11b, a marker of late differentiation of myeloid cells, in Allergin-1⁺ AML cells is significantly lower than that of control Allergin-1-cells, (n=3), *p<0.05 (FIGS. 4C and 4D).

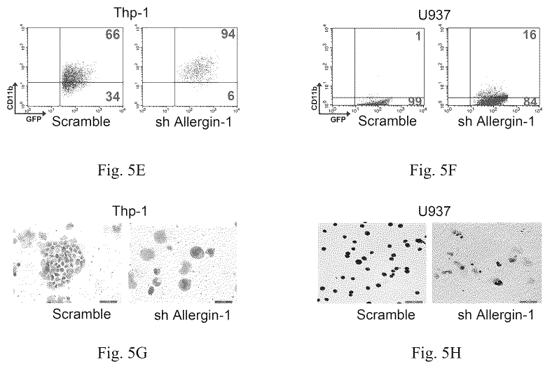

The effect of specific interference sequence or non-specific interference sequence targeting human Allergin-1 gene on the proliferation, differentiation and in vivo engraftment of AML cells was detected. It was found that the Allergin-1 knockdown group significantly inhibits the proliferation of AML cells, and significantly improves the expression level of CD11b in AML cells, compared with the control group (n=3), *p<0.05 (FIGS. 5A-5H).

### Example 4

### Detection of the Effect of IFN-γ on the Expression of Allergin-1 and Proliferation of AML Cells

### Experimental Method:

HL-60, Thp-1, MV4-11 and U937 cells (3×10⁴ cells) were cultured in DMEM complete medium or DMEM complete medium containing 30 ng/ml recombinant human IFN-γ (Catalog #300-02, Peprotech) for 2 days. Then, the effect of IFN-γ on the expression of Allergin-1 and proliferation of AML cells was detected by flow cytometry and cell counting. Furthermore, HL-60, Thp-1, MV4-11 and U937 cells were cultured in DMEM complete medium containing IFN-γ (30 ng/ml) for 2 days. Then 1×10³ Allergin-1 positive or Allergin-1 negative cells were sorted by flow cytometry, and further cultured in DMEM complete medium for 6 days. The effect of IFN-γ pretreatment of cells on the proliferation of AML was detected by cell counting.

### Experimental Results:

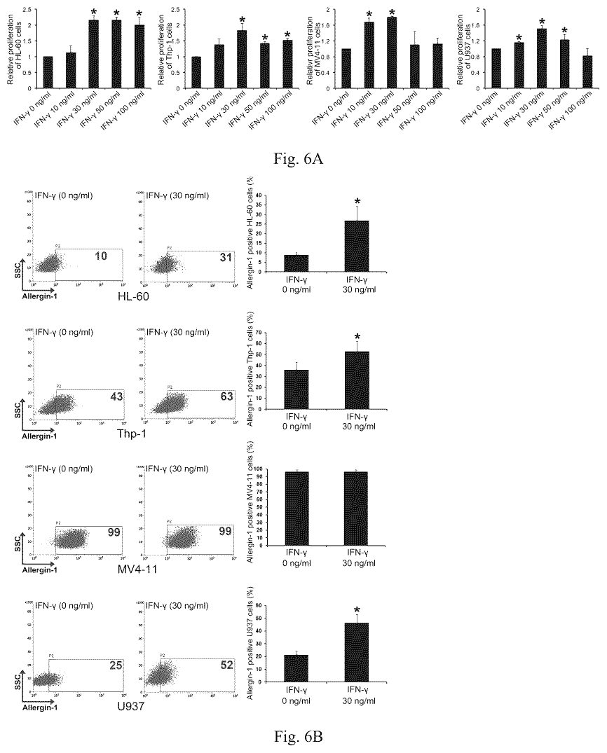

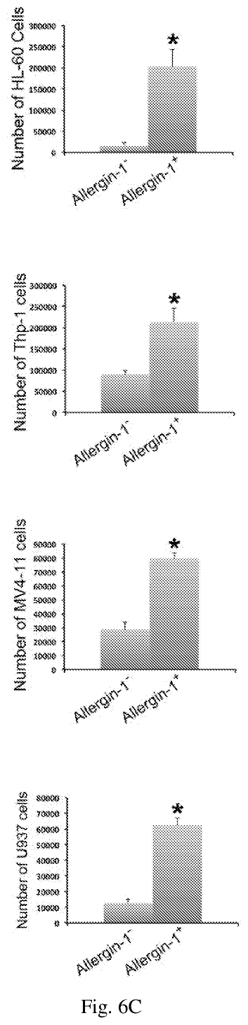

The effect of IFN-γ on the proliferation of AML cells and the expression of Allergin-1 was detected. It was found that IFN-γ promotes the proliferation of AML cells (FIG. 6A) and the expression level of Allergin-1 (FIG. 6B), (n=3), *p<0.05, and after IFN-γ pretreatment of AML cells, the proliferation ability of Allergin-1⁺ cells is significantly higher than that of the control group Allergin-1⁻ cells (FIG. 6C), (n=3), *p<0.05.

### Example 5

### Detection of the Effect of Allergin-1 on Engraftment of AML Cells In Vivo

### Experimental Method:

shRNA pretreatment of cells for xenotransplantation experiments: GFP-positive Allergin-1-knockdown Thp-1 cells were transplanted into NPG mice through retro-orbital injection (Beijing Vital River Laboratory Animal Technology Co., Ltd.), and an experimental group and a negative control group were set up, with 6 animals each group. The experimental group was inoculated with GFP-positive Thp-1 cells pretreated with Allergin-1 shRNA at 1×10⁶ cells/mouse. The negative control group was inoculated with GFP-positive Thp-1 cells pretreated with a non-specific interference sequence at 1×10⁶ cells/mouse. Twelve weeks after transplantation, the proportion of GFP-positive cells in spleen and liver cells of mice was measured by flow cytometry to evaluate the engraftment ability of the transplanted cells.

### Experimental Results:

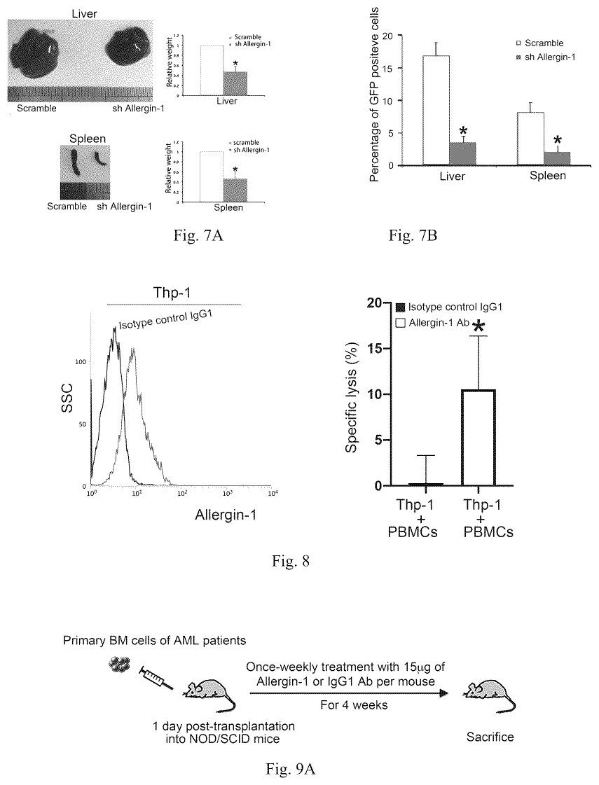

It was further found that the weight of liver and spleen of mice transplanted with Allergin-1 knockdown Thp-1 cells is significantly lower than that of the control group (FIG. 7A), (n=3), *p<0.05; and the engraftment ability of Allergin-1 knockdown Thp-1 cells in liver and spleen cells of mice is significantly lower than that of the control group (FIG. 7B), (n=3), *p<0.05.

### Example 6

### Evaluation of the Feasibility of Allergin-1 Antibody to Selectively Eliminate AML Cells

### Experimental Method:

ADCC (antibody-dependent cell-mediated cytotoxicity) effect of Allergin-1 antibody was determined by using PBMCs as effector cells and human AML cell line Thp-1 cells (ATCC (American type culture collection), TIB-202) as target cells. PBMCs were purified from healthy volunteers by Ficoll-Hypaque density gradient centrifugation (Pharmacia Biotech, Uppsala, Sweden), and incubated in DMEM complete media containing 15 ng/ml of recombinant human IL-2 (Catalog #200-02, Peprotech) for 18 hours at 37° C. Thp-1 cells were collected as target cells. The Allergin-1 antibody as the experimental group and the IgG1 antibody as the isotype control group were respectively added to the prepared target cells at a final concentration of 2 μg/ml, and incubated at 37° C. for 30 min. The target cell suspension was added to a 96-well U-shaped plate at 50 μl/well. The effector cells were added with an effector cell/target cell ratio of 10:1 at 50 μl/well, to a total volume of 100 μl/well. After culture in an incubator at 37° C., 5% CO₂ for 5 hours, color development and reading were performed using the CellTiter 96® AQueous non-radioactive cell proliferation detection kit (CytoTox 96® Non-Radioactive Cytotoxicity Assay, Catalog number: G1780, Promega).

In vivo detection of the effect of Allergin-1 antibody on the hematopoietic reconstitution ability of CD34⁺ cells from healthy cord blood or on the growth of bone marrow cells from patients with primary AML in NOD/SCID mice (Beijing Vital River Laboratory Animal Technology Co., Ltd.): CD34⁺ cells from healthy cord blood (1×10⁶ cells/mouse) or bone marrow cells from patients with primary AML (1×10⁶ cells/mouse) were transplanted into NOD/SCID mice through retro-orbital injection, respectively. After 24 hours of the transplantation, the mice were treated with Allergin-1 or IgG antibody (intravenous injection), with once a week. Each mouse was treated with 15 μg of antibody each time for 4 weeks. Five weeks after transplantation, the proportion of hCD45⁺ cells (Clone #J33, Beckman Coulter) in bone marrow cells of mice and the proportion of CD34⁺CD38⁻, CD34⁺, Allergin-1⁺, CD33⁺(Clone #HIM3-4, eBioscience), CD19⁺ (Clone #HIB19, eBioscience) or Allergin-1⁺CD33⁺ cells in hCD45⁺ cells were detected by flow cytometry.

### Experimental Results:

In vitro, after mixing of Thp-1 cells of AML cell line and Allergin-1 antibody with PBMCs, the cytotoxicity of PBMCs to AML cells mediated by Allergin-1 antibody (ADCC) was detected. It was found that the cytotoxicity of PBMCs to AML cells mediated by Allergin-1 antibody is significantly higher than that of the control group (FIG. 8), (n=3), *p<0.05.

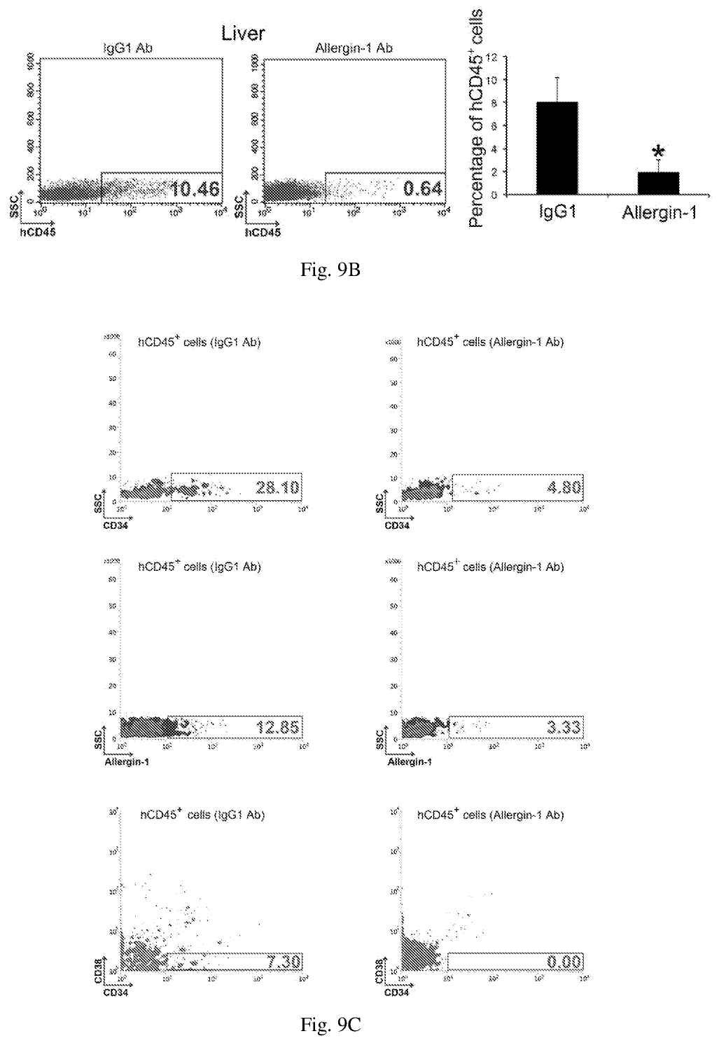

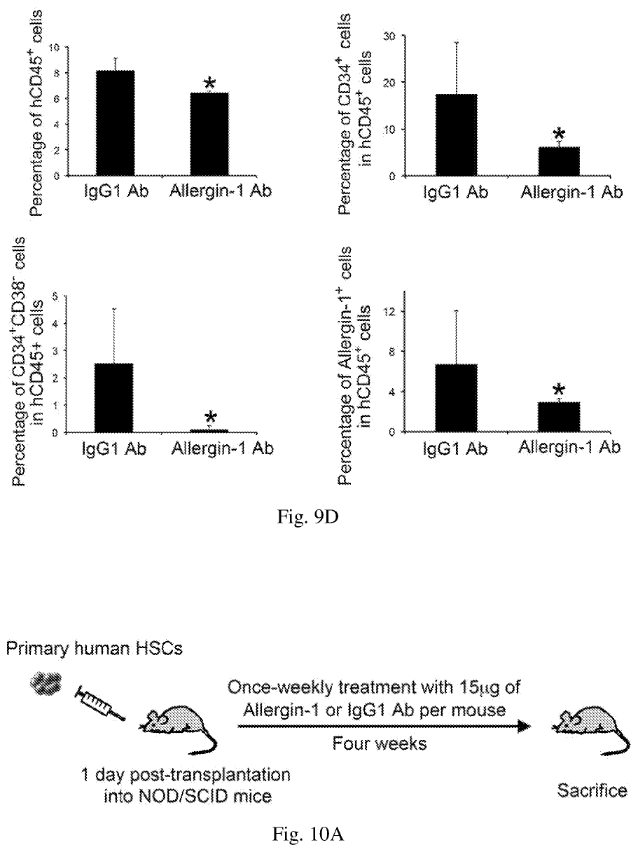

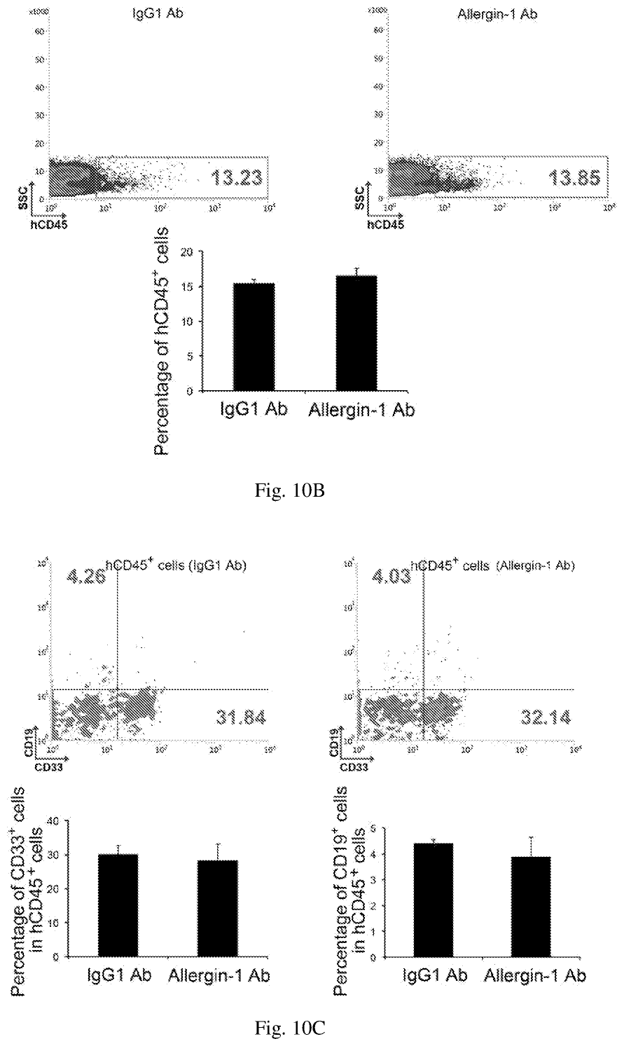

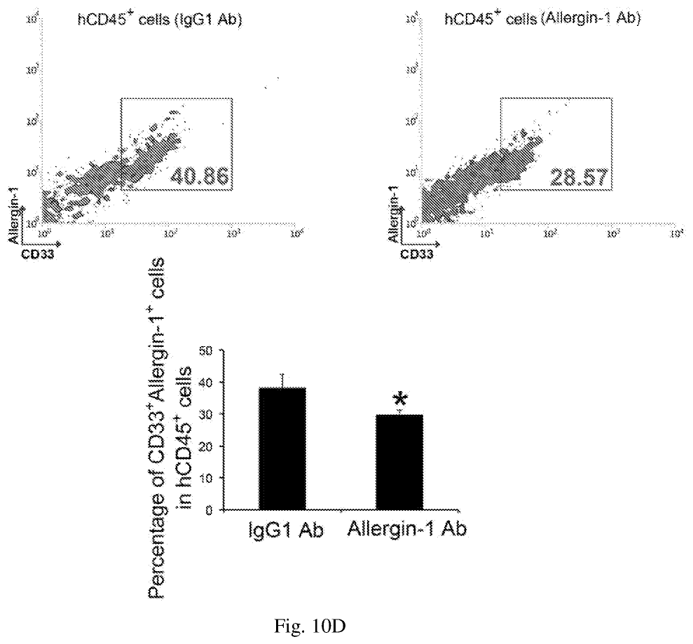

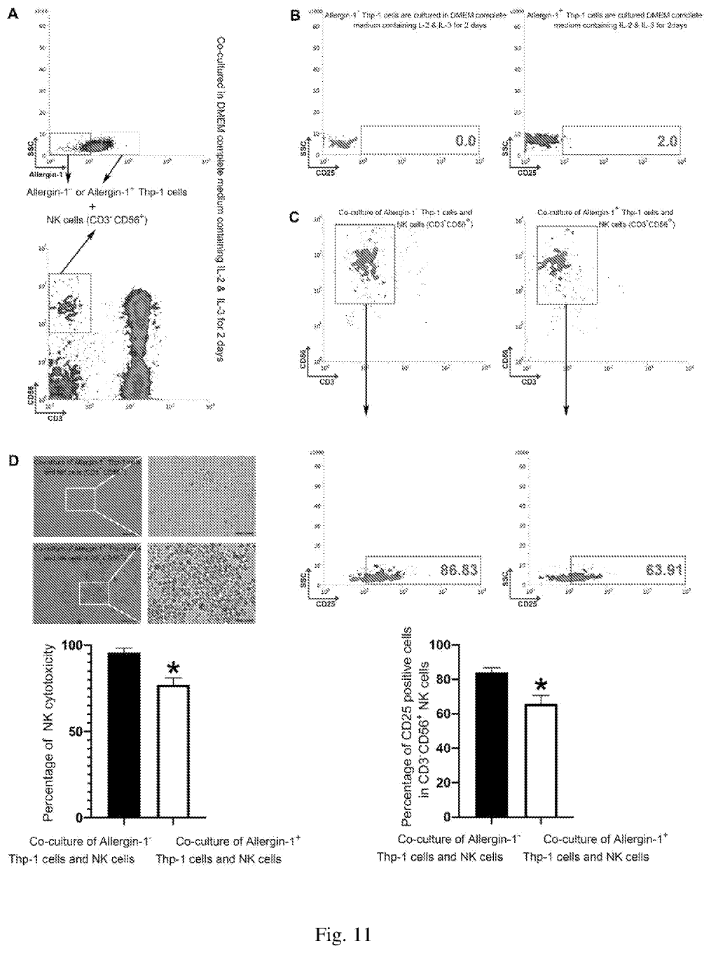

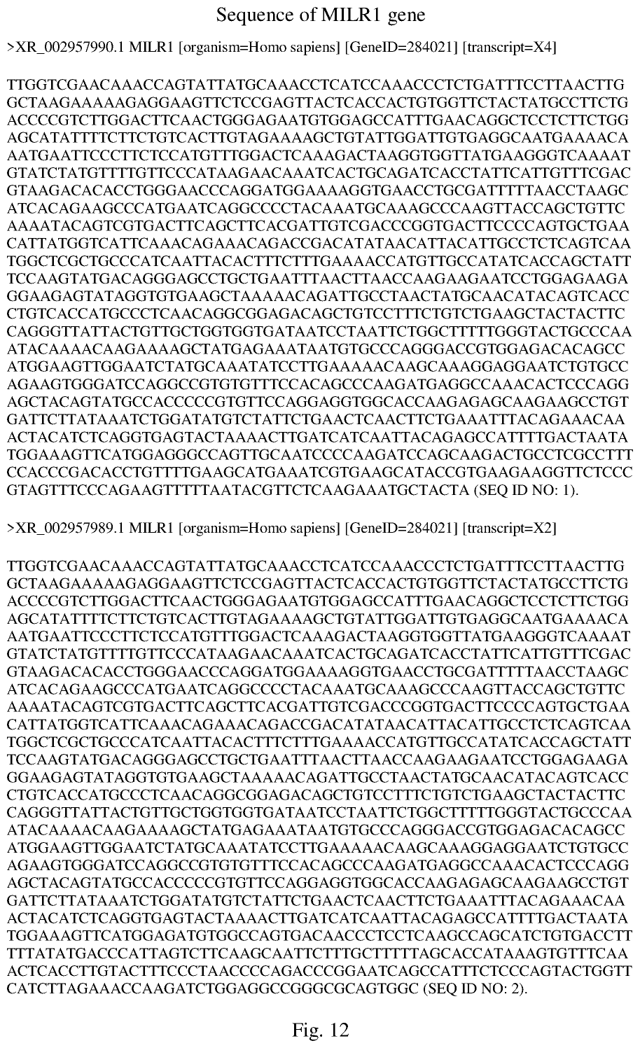

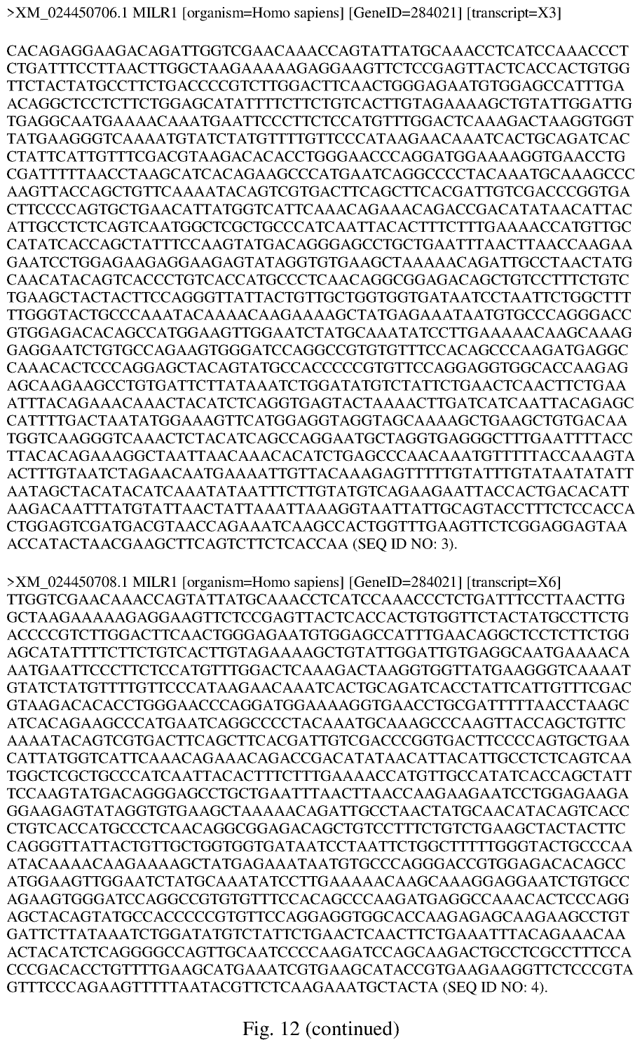

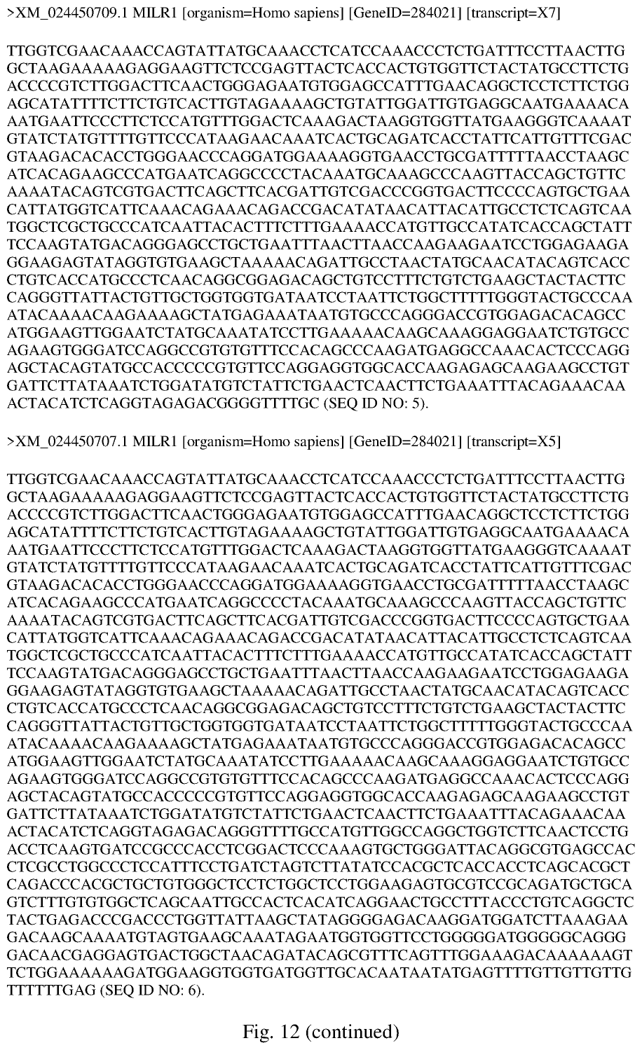

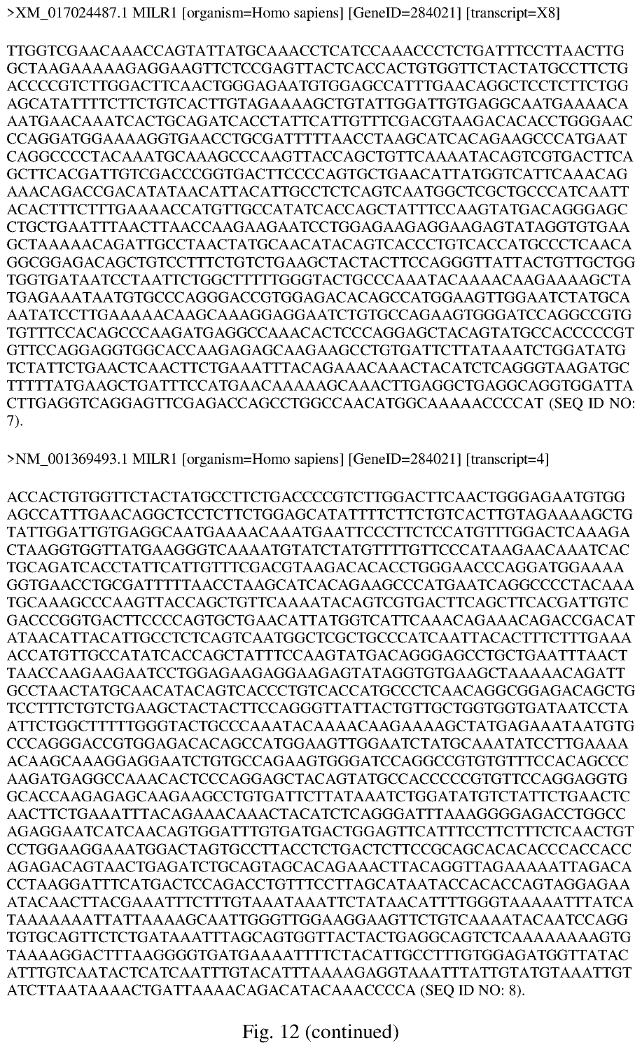

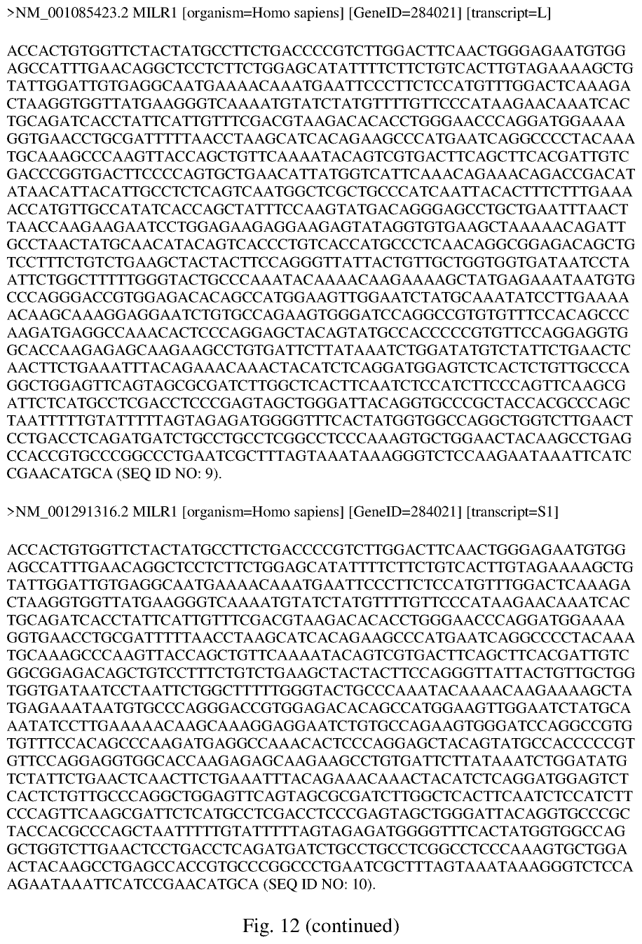

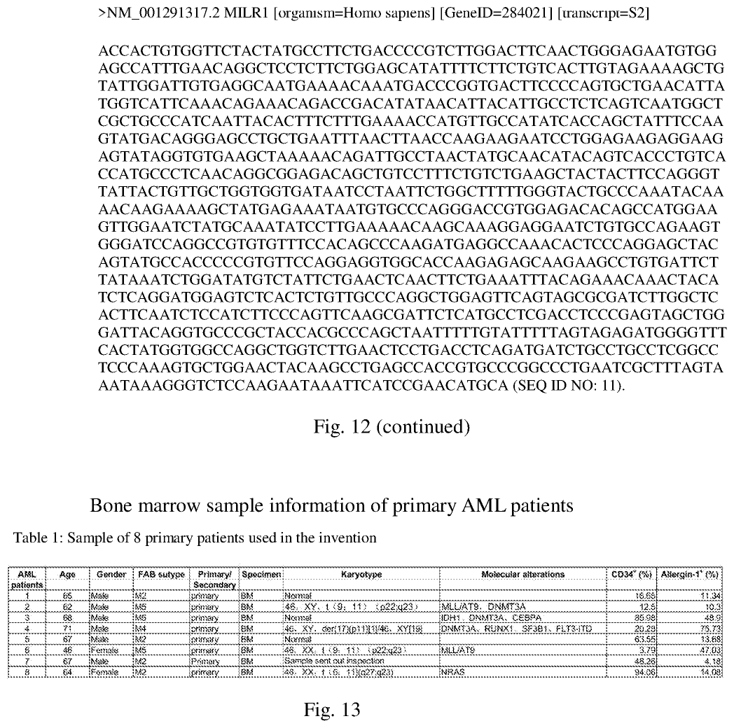

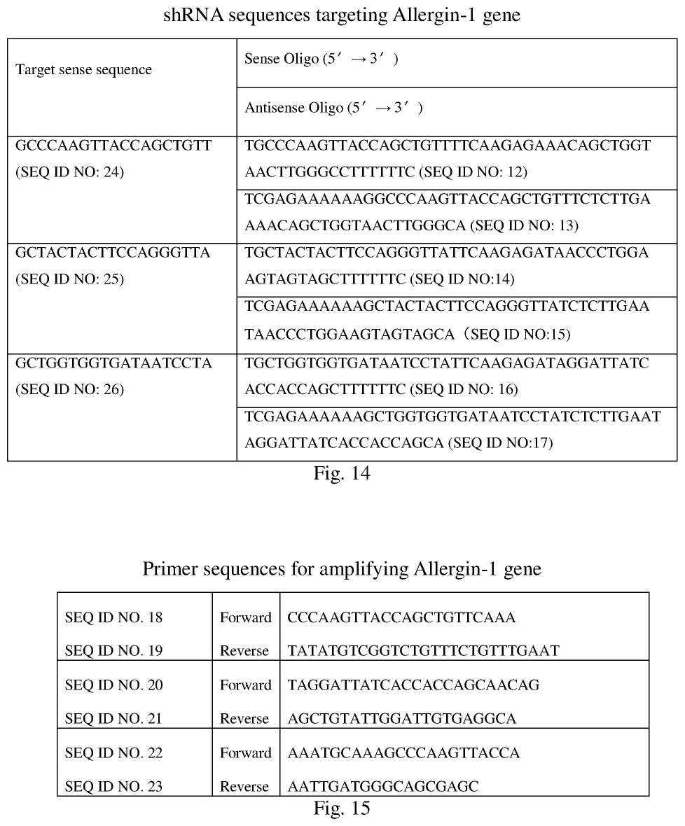

Allergin-1 is highly expressed in LSCs and monocytic AML cells, but is not expressed in normal HSCs and lowly expressed in normal monocytes (FIGS. 2A-2D). Therefore, the cytotoxicity of immune effector cells to bone marrow cells of patients with primary AML mediated by Allergin-1 antibody was detected in vivo. The bone marrow cells of patients with primary AML were transplanted into NOD/SCID mice through retro-orbital injection to establish a human-xenograft AML mouse model. Model mice were treated with IgG1 or Allergin-1 antibody. The proportions of human AML cells in liver or bone marrow of mice in different treatment groups were measured by flow cytometry after 5 weeks of transplantation (FIG. 9A). It was found that the proportion of human CD45⁺ cells in mouse liver cells in the Allergin-1 antibody treatment group is significantly lower than that in the control IgG1 antibody treatment group (FIG. 9B). It was further found that the proportions of human CD45⁺ cells in mouse bone marrow, CD34⁺AML cells in human CD45⁺ cells, CD34⁺CD38⁻ in human CD45⁺ cells and Allergin-1⁺ cells in human CD45⁺ cells in the Allergin-1 antibody treatment group are significantly lower than those in the IgG1 antibody treatment group (FIGS. 9C and 9D). Especially in the Allergin-1 antibody treatment group, human CD34⁺CD38⁻ LSCs can hardly be detected in mouse bone marrow cells (bottom panel of FIG. 9C to bottom left panel of FIG. 9D), (N=3), *p<0.05. It is suggested that the Allergin-1 antibody can fundamentally eliminate LSCs that are the root cause of AML. The Allergin-1 antibody does not affect the in vivo hematopoietic reconstitution and engraftment ability of CD34⁺ cells from cord blood (FIGS. 10A-10D), (n=3), *p<0.05, indicating the safety of the Allergin-1 antibody.

### Example 7

### Detection of the Effect of Allergin-1 on Activity and Cytotoxicity of NK Cells

### Experimental Method:

The effect of Allergin-1 on activity of NK cells was detected by co-culture of Allergin-1⁺ or Allergin-1⁻ AML cells with CD3⁺CD56⁺ NK cells derived from peripheral blood mononuclear cells of healthy human. CD3⁺CD56⁺ NK cells were separated from peripheral blood mononuclear cells of healthy human and adjusted to the cell concentration of 1×10⁶ cells/ml. Allergin-1⁺ or Allergin-1⁺ Thp-1 cells were collected and adjusted to the cell concentration of 2×10⁵ cells/ml. 100 μl of NK cells were mixed thoroughly with 100 μl of Allergin-1⁺or Allergin-1⁺ Thp-1 cell suspension and then added to a 96-well U-shaped plate with a total volume of 200 μl/well. After culture in DMEM complete medium containing 100 ng/ml recombinant human IL-2 and 1 ng/ml recombinant human IL-3 (Catalog #200-03, Peprotech) for 2 days, CD25, a marker of activated NK cells, was detected by flow cytometry. The number of viable cells was determined by measuring their metabolic activity using the Cell Counting Kit-8 (CCK8, Sangon Biotech, Shanghai, China). The antibodies used are anti-human CD25-PE (Clone: 1D4B, BioLegend), anti-human CD56-PC5.5 (Clone: PK136, BioLegend) and anti-human CD3e-FITC (Clone: 145-2C11, eBioscience), respectively.

### Experimental Results:

After co-culture of Allergin-1⁺ or Allergin-1⁺ Thp-1 cells with CD3⁺CD56⁺ NK cells derived from peripheral blood mononuclear cells of healthy human in vitro for 2 days, the effect of Allergin-1 on activity of NK cells was detected. It was found that the expression level of CD25 (a marker of activated NK cells) in NK cells co-cultured with Allergin-1⁻ Thp-1 cells is significantly higher than that in NK cells co-cultured with Allergin-1⁺Thp-1 cells, and the cytotoxicity of NK cells co-cultured with Allergin-1⁺ Thp-1 cells is significantly higher than that of NK cells co-cultured with Allergin-1⁺ Thp-1 cells (FIG. 11), (n=3), *p<0.05. The above findings suggest that the activation of NK cells is related to the expression of Allergin-1 in co-cultured AML cells. Therefore, reducing the expression of Allergin-1 or/and the activity of Allergin-1 in AML cells facilitates more effectively eliminating AML cells.

All features disclosed in this specification can be combined in any combination. Each feature disclosed in this specification can be replaced by an alternative feature having the same, equivalent or similar purpose. Therefore, unless explicitly stated otherwise, each feature disclosed is merely an example of a series of equivalent or similar features.

Based on the above description, those skilled in the art can easily identify the basic features of the present invention, and can make various changes and modifications to the present invention to adapt to various uses and conditions without departing from the spirit and scope of the present invention. Therefore, other embodiments are also within the scope of the appended claims.

1. A method for identification of LSCs, comprising the identification of LSCs by detecting expression or activity of biomarker Allergin-1 in a subject or a sample from a subject;
the expression level or activity of said Allergin-1 in hematopoietic stem cells is at least 3 times of that in normal hematopoietic stem cells, which is an indication that the hematopoietic stem cells are LSCs.

2. A method for diagnosis, disease course monitoring and prognosis of leukemia, or determination of risk of developing leukemia, or determination of severity of leukemia, comprising detecting the expression or activity of the biomarker Allergin-1 in hematopoietic stem cells or monocytes in the subject or the sample from the subject;
the expression level or activity of said Allergin-1 in hematopoietic stem cells is at least 3 times of that in normal hematopoietic stem cells, which is an indication that the hematopoietic stem cells are LSCs
the expression level or activity of said Allergin-1 in monocytes is at least 2 times of that in normal monocytes, which is an indication that the monocytes are monocytic AML cell.

3-4. (canceled)

5. A method for treatment of leukemia, comprising administering to a subject a therapeutically effective amount of an inhibitor/antagonist for expression or activity of Allergin-1.

6-19. (canceled)

20. The method of claim 5, wherein the leukemia is acute myeloid leukemia AML.

21. (canceled)

22. The method of claim 1, wherein the Allergin-1 is combined with at least one biomarker, the combination is Allergin-1⁺CD34⁺CD38⁻.

23-25. (canceled)

26. The method of claim 5, wherein the treatment is targeted therapy and/or immunotherapy.

27. The method of claim 5, wherein the inhibitor and/or antagonist refers to the substance that reduces activity of Allergin-1 protein, reduces stability of Allergin-1 gene or protein, down regulates expression of Allergin-1, reduces effective acting time of Allergin-1 protein, or inhibits transcription and translation of Allergin-1.

28. The method of claim 5, wherein the inhibitor/antagonist for expression or activity of Allergin-1 is selected from nucleic acid inhibitor, protein inhibitor, proteolytic enzyme, protein binding molecule; and combination thereof.

29. The method of claim 5, wherein the inhibitor/antagonist for expression or activity of Allergin-1 is selected from interfering molecules that target Allergin-1 or its transcript and are capable of inhibiting expression or transcription of Allergin-1 gene.

30. The method of claim 28, wherein the protein binding molecules are selected from antibodies or ligands capable of inhibiting the activity of Allergin-1 protein; competitors for Allergin-1 ligand binding site, including Allergin-1 receptors and ligand binding fragments thereof, soluble truncated Allergin-1 receptors, soluble Allergin-1 receptor fusion proteins, Allergin-1 fusion proteins or ligand fusion proteins containing Fc portion of IgG immunoglobulins; peptidomimetics; peptide inhibitors; small molecule compounds; and combination thereof.

31. The method of claim 5 wherein the inhibitor/antagonist for expression or activity of Allergin-1 is shRNA; the shRNA has a sequence shown in SEQ ID NO: 12, 13, 14, 15, 16 or 17.

32. The method of claim 5, wherein the inhibitor/antagonist for expression or activity of Allergin-1 is an antibody that specifically binds to Allergin-1.

33. The method of claim 5, wherein the inhibitor/antagonist for expression or activity of Allergin-1 is administered at about 0.01 mg/kg to about 100 mg/kg, about 0.01 mg/kg-0.2 mg/kg, about 0.1 mg/kg-10 mg/kg, about 0.5 mg/kg-5 mg/kg, or about 1 mg/kg to about 2 mg/kg

34. The method of claim 5, wherein the inhibitor/antagonist for expression or activity of Allergin-1 is administered by intravenous infusion.

35. The method of claim 5, wherein the treatment eliminates LSCs and/or monocytic AML cells and/or improves activity of NK cells derived from normal monocytes.

36. The method of claim 5, comprising administering to the subject a therapeutically effective amount of the inhibitor/antagonist for expression or activity of Allergin-1 in combination with one or more therapeutic agents.

37. The method of claim 36, wherein the one or more therapeutic agents are IFN-γ.

38. The method of claim 20, wherein the AML is of an M2, M4, or M5 subtype.

39. The method of claim 29, wherein the inhibitor/antagonist is selected from shRNA (small hairpin RNA), small interfering RNA (siRNA), dsRNA, microRNA, zinc finger, and CRISPR/Cas9.

40. The method of claim 33, wherein the unit dose of the inhibitor/antagonist for expression or activity of Allergin-1 is the unit dose of about 1 mg to about 100 mg, about 5 mg-50 mg, about 10 mg-40 mg, 15 mg-30 mg, or 20 mg-25 mg.

41. The method of claim 34, during the treatment cycle, the inhibitor/antagonist for expression or activity of Allergin-1 is administered once a month, once every two weeks, once, twice or more times a week, once, twice or more times a day; and a proteasome inhibitor is administered once a month, once every two weeks, once, twice or more times a week, once, twice or more times a day.


SEQUENCE LISTING (26 sequences)

SEQ ID NO: 1 - DNA, 1419 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggtgagtactaaaacttgatcatcaattacagagccattttgactaatatggaaagttcatggagggccagttgcaatccccaagatccagcaagactgcctcgcctttccacccgacacctgttttgaagcatgaaatcgtgaagcataccgtgaagaaggttctcccgtagtttcccagaagtttttaatacgttctcaagaaatgctacta

SEQ ID NO: 2 - DNA, 1475 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggtgagtactaaaacttgatcatcaattacagagccattttgactaatatggaaagttcatggagatgtggccagtgacaaccctcctcaagccagcatctgtgacctttttatatgacccattagtcttcaagcaattctttgctttttagcaccataaagtgtttcaaactcaccttgtactttccctaaccccagacccggaatcagccatttctcccagtactggttcatcttagaaaccaagatctggaggccgggcgcagtggc

SEQ ID NO: 3 - DNA, 1709 bp, Artificial Sequence
  Sequence of MILR1 gene
cacagaggaagacagattggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggtgagtactaaaacttgatcatcaattacagagccattttgactaatatggaaagttcatggaggtaggtagcaaaagctgaagctgtgacaatggtcaagggtcaaactctacatcagccaggaatgctaggtgagggctttgaattttaccttacacagaaaggctaattaacaaacacatctgagcccaacaaatgtttttaccaaagtaactttgtaatctagaacaatgaaaattgttacaaagagtttttgtatttgtataatatattaatagctacatacatcaaatataatttcttgtatgtcagaagaattaccactgacacattaagacaatttatgtattaactattaaattaaaggtaattattgcagtacctttctccaccactggagtcgatgacgtaaccagaaatcaagccactggtttgaagttctcggaggagtaaaccatactaacgaagcttcagtcttctcaccaa

SEQ ID NO: 4 - DNA, 1356 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggggccagttgcaatccccaagatccagcaagactgcctcgcctttccacccgacacctgttttgaagcatgaaatcgtgaagcataccgtgaagaaggttctcccgtagtttcccagaagtttttaatacgttctcaagaaatgctacta

SEQ ID NO: 5 - DNA, 1224 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggtagagacggggttttgc

SEQ ID NO: 6 - DNA, 1741 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggtagagacagggttttgccatgttggccaggctggtcttcaactcctgacctcaagtgatccgcccacctcggactcccaaagtgctgggattacaggcgtgagccacctcgcctggccctccatttcctgatctagtcttatatccacgctcaccacctcagcacgctcagacccacgctgctgtgggctcctctggctcctggaagagtgcgtccgcagatgctgcagtctttgtgtggctcagcaattgccactcacatcaggaactgcctttaccctgtcaggctctactgagacccgaccctggttattaagctataggggagacaaggatggatcttaaagaagacaagcaaaatgtagtgaagcaaatagaatggtggttcctgggggatgggggcaggggacaacgaggagtgactggctaacagatacagcgtttcagtttggaaagacaaaaaagttctggaaaaaagatggaaggtggtgatggttgcacaataatatgagttttgttgttgttgttttttgag

SEQ ID NO: 7 - DNA, 1247 bp, Artificial Sequence
  Sequence of MILR1 gene
ttggtcgaacaaaccagtattatgcaaacctcatccaaaccctctgatttccttaacttggctaagaaaaagaggaagttctccgagttactcaccactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcagggtaagatgctttttatgaagctgatttccatgaacaaaaagcaaacttgaggctgaggcaggtggattacttgaggtcaggagttcgagaccagcctggccaacatggcaaaaaccccat

SEQ ID NO: 8 - DNA, 1716 bp, Artificial Sequence
  Sequence of MILR1 gene
accactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcagggatttaaaggggagacctggccagaggaatcatcaacagtggatttgtgatgactggagttcatttccttctttctcaactgtcctggaaggaaatggactagtgccttacctctgactcttccgcagcacacacccaccaccagagacagtaactgagatctgcagtagcacagaaacttacaggttagaaaaattagacacctaaggatttcatgactccagacctgtttccttagcataataccacaccagtaggagaaatacaacttacgaaatttctttgtaaataaattctataacattttgggtaaaaatttatcataaaaaaattattaaaagcaattgggttggaaggaagttctgtcaaaatacaatccaggtgtgcagttctctgataaatttagcagtggttactactgaggcagtctcaaaaaaaagtgtaaaaggactttaaggggtgatgaaaattttctacattgcctttgtggagatggttatacatttgtcaatactcatcaatttgtacatttaaaagaggtaaatttattgtatgtaaattgtatcttaataaaactgattaaaacagacatacaaacccca

SEQ ID NO: 9 - DNA, 1448 bp, Artificial Sequence
  Sequence of MILR1 gene
accactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggatggagtctcactctgttgcccaggctggagttcagtagcgcgatcttggctcacttcaatctccatcttcccagttcaagcgattctcatgcctcgacctcccgagtagctgggattacaggtgcccgctaccacgcccagctaatttttgtatttttagtagagatggggtttcactatggtggccaggctggtcttgaactcctgacctcagatgatctgcctgcctcggcctcccaaagtgctggaactacaagcctgagccaccgtgcccggccctgaatcgctttagtaaataaagggtctccaagaataaattcatccgaacatgca

SEQ ID NO: 10 - DNA, 1163 bp, Artificial Sequence
  Sequence of MILR1 gene
accactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgaattcccttctccatgtttggactcaaagactaaggtggttatgaagggtcaaaatgtatctatgttttgttcccataagaacaaatcactgcagatcacctattcattgtttcgacgtaagacacacctgggaacccaggatggaaaaggtgaacctgcgatttttaacctaagcatcacagaagcccatgaatcaggcccctacaaatgcaaagcccaagttaccagctgttcaaaatacagtcgtgacttcagcttcacgattgtcggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggatggagtctcactctgttgcccaggctggagttcagtagcgcgatcttggctcacttcaatctccatcttcccagttcaagcgattctcatgcctcgacctcccgagtagctgggattacaggtgcccgctaccacgcccagctaatttttgtatttttagtagagatggggtttcactatggtggccaggctggtcttgaactcctgacctcagatgatctgcctgcctcggcctcccaaagtgctggaactacaagcctgagccaccgtgcccggccctgaatcgctttagtaaataaagggtctccaagaataaattcatccgaacatgca

SEQ ID NO: 11 - DNA, 1178 bp, Artificial Sequence
  Sequence of MILR1 gene
accactgtggttctactatgccttctgaccccgtcttggacttcaactgggagaatgtggagccatttgaacaggctcctcttctggagcatattttcttctgtcacttgtagaaaagctgtattggattgtgaggcaatgaaaacaaatgacccggtgacttccccagtgctgaacattatggtcattcaaacagaaacagaccgacatataacattacattgcctctcagtcaatggctcgctgcccatcaattacactttctttgaaaaccatgttgccatatcaccagctatttccaagtatgacagggagcctgctgaatttaacttaaccaagaagaatcctggagaagaggaagagtataggtgtgaagctaaaaacagattgcctaactatgcaacatacagtcaccctgtcaccatgccctcaacaggcggagacagctgtcctttctgtctgaagctactacttccagggttattactgttgctggtggtgataatcctaattctggctttttgggtactgcccaaatacaaaacaagaaaagctatgagaaataatgtgcccagggaccgtggagacacagccatggaagttggaatctatgcaaatatccttgaaaaacaagcaaaggaggaatctgtgccagaagtgggatccaggccgtgtgtttccacagcccaagatgaggccaaacactcccaggagctacagtatgccacccccgtgttccaggaggtggcaccaagagagcaagaagcctgtgattcttataaatctggatatgtctattctgaactcaacttctgaaatttacagaaacaaactacatctcaggatggagtctcactctgttgcccaggctggagttcagtagcgcgatcttggctcacttcaatctccatcttcccagttcaagcgattctcatgcctcgacctcccgagtagctgggattacaggtgcccgctaccacgcccagctaatttttgtatttttagtagagatggggtttcactatggtggccaggctggtcttgaactcctgacctcagatgatctgcctgcctcggcctcccaaagtgctggaactacaagcctgagccaccgtgcccggccctgaatcgctttagtaaataaagggtctccaagaataaattcatccgaacatgca

SEQ ID NO: 12 - DNA, 56 bp, Artificial Sequence
  shRNA sequence
tgcccaagttaccagctgttttcaagagaaacagctggtaacttgggccttttttc

SEQ ID NO: 13 - DNA, 60 bp, Artificial Sequence
  shRNA sequence
tcgagaaaaaaggcccaagttaccagctgtttctcttgaaaacagctggtaacttgggca

SEQ ID NO: 14 - DNA, 55 bp, Artificial Sequence
  shRNA sequence
tgctactacttccagggttattcaagagataaccctggaagtagtagcttttttc

SEQ ID NO: 15 - DNA, 59 bp, Artificial Sequence
  shRNA sequence
tcgagaaaaaagctactacttccagggttatctcttgaataaccctggaagtagtagca

SEQ ID NO: 16 - DNA, 55 bp, Artificial Sequence
  shRNA sequence
tgctggtggtgataatcctattcaagagataggattatcaccaccagcttttttc

SEQ ID NO: 17 - DNA, 59 bp, Artificial Sequence
  shRNA sequence
tcgagaaaaaagctggtggtgataatcctatctcttgaataggattatcaccaccagca

SEQ ID NO: 18 - DNA, 22 bp, Artificial Sequence
  Primer sequence for amplifying Allergin-1 gene??Forward??
cccaagttaccagctgttcaaa

SEQ ID NO: 19 - DNA, 27 bp, Artificial Sequence
  Primer sequence for amplifying Allergin-1 gene??Reverse??
tatatgtcggtctgtttctgtttgaat

SEQ ID NO: 20 - DNA, 24 bp, Artificial Sequence
  Primer sequence for amplifying Allergin-1 gene??Forward??
taggattatcaccaccagcaacag

SEQ ID NO: 21 - DNA, 22 bp, Artificial Sequence
  Primer sequence for amplifying Allergin-1 gene??Reverse??
agctgtattggattgtgaggca

SEQ ID NO: 22 - DNA, 22 bp, Artificial Sequence
  Primer sequence for amplifying Allergin-1 gene??Forward??
aaatgcaaagcccaagttacca

SEQ ID NO: 23 - DNA, 18 bp, Artificial Sequence
  Primer sequence for amplifying Allergin-1 gene??Reverse??
aattgatgggcagcgagc

SEQ ID NO: 24 - DNA, 19 bp, Artificial Sequence
  Target sense sequence of Allergin-1 gene
gcccaagttaccagctgtt

SEQ ID NO: 25 - DNA, 19 bp, Artificial Sequence
  Target sense sequence of Allergin-1 gene
gctactacttccagggtta

SEQ ID NO: 26 - DNA, 19 bp, Artificial Sequence
  Target sense sequence of Allergin-1 gene
gctggtggtgataatccta